(19) United States
(12) Patent Application Publication
(10) Pub. No.: US 2012/0316234 A1
(43) Pub. Date: Dec. 13, 2012

(54) ANTHRAQUINONE -BASED COMPOUND, THE SYNTHESIS PROCESSES AND THE APPLICATION THEREOF

(75) Inventor: Hsu-Shan HUANG, Taipei (TW)

(73) Assignee: NATIONAL DEFENSE MEDICAL CENTER, Taipei (TW)

(21) Appl. No.: 13/268,190

(22) Filed: Oct. 7, 2011

(30) Foreign Application Priority Data
Jun. 10, 2011 (TW) 100120369

(51) Int. Cl. A61K 31/216; A61P 35/00; C07C 229/40; C07C 231/12; C07C 233/01; A61K 31/167

(52) U.S. Cl. 514/510; 564/211; 514/628; 560/44

(57) ABSTRACT

The present invention is related to an anthraquinone-based compound represented by compound (I)

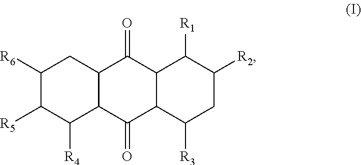

wherein the R1, R2, R3, R4, R5 or R6 is selected by the group of H, amide, amino-amide, acyl, chloroacetamide, chloropropionamido, a substituted chloroacetamide and a substituted chloropropionamido. The present invention is also related to the method of producing the anthraquinone-based compound and the application thereof.

### CROSS-REFERENCE TO RELATED APPLICATIONS

This Non-provisional application claims priority under 35 U.S.C. §119(a) on Patent Application No(s). 100120369 filed in Taiwan, Republic of China Jun. 10, 2011, the entire contents of which are hereby incorporated by reference.

### FIELD OF THE INVENTION

This invention relates to an anthraquinone-based compound, the synthesis processes and the application thereof.

### BACKGROUND OF THE INVENTION

The anthracycline antibiotics daunorubicin, doxorubicin, mitoxantrone and ametantrone have been used widely as anticancer drugs for many decades, but their cardio toxicity limits their clinical use. The detailed mechanism of how these compounds, all of which contain an anthraquinone-linked pharmacophore, remained elusive although they might rendered their effects through forming complexes with the DNA base pairs or G-quadruplex. The mechanism includes: (1) the compounds form a complex with topoisomerase first, the complex links to DNA and cuts out of DNA. When DNA double-helix is broken, the complex could stay on DNA. The DNA replication would stop on this site when the cell grows, and the cell would stop growth. (2) The compounds form a complex with DNA first, then form a complex with topoisomerase, the complex links to DNA and cuts out of DNA. When DNA double-helix is broken, the complex could stay on DNA. The DNA replication would stop on this site when the cell grows, and the cell would stop growth because the DNA is cut out.

On the other hand, the cancer therapeutic effect of mitoxantrone induced Collier and Neidle mimicked mitoxantrone by substituting the 1, 4 carbon and found the 1,4-daminoanthraquinone deritives' inhibition effect to L1210 cancer cells in 1988. Moreover, Gatto found that the small amino-acid dual substituted 1,4-anthraquinone has higher anti-cancer activity than single substituted 1,4-anthraquinone. Besides, the longer the side chain is the lower the anti-cancer activity is and the higher affinity to GC segments of DNA is.

Therefore, the aim of the present invention is to inhibit telomerase for the anti-cancer effect by developing anthraquinone compounds.

### SUMMARY OF THE INVENTION

As the reason mentioned above, the present invention provides an anthraquinone-based compound represented by compound (I)

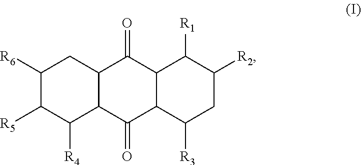

wherein the R1, R2, R3, R4, R5 or R6 is selected from the group consisting of H, amide, amino-amide, acyl, chloroacetamide, chloropropionamide, a substituted acetamide and a substituted propionamide;

wherein if the R1 and R3 are selected from the group consisting of non-substituted or substituted acetamide and non-substituted or substituted propionamido, the R2, R4, R5 and R6 are H;

wherein if the R1 and R4 are selected from the group consisting of non-substituted or substituted acetamide and non-substituted or substituted propionamide, the R2, R3, R5 and R6 are H;

wherein if the R2 and R5 are selected from the group consisting of non-substituted or substituted acetamide and non-substituted or substituted propionamide, the R1, R3, R4 and R6 are H;

wherein if the R2 and R6 are selected from the group consisting of non-substituted or substituted acetamide and non-substituted or substituted propionamide, the R1, R3, R4 and R5 are H.

The invention also provides a method of synthesizing an anthraquinone-based compound, which includes substituting R1, R2, R3, R4, R5 or R6 by non-substituted or substituted acetamide and non-substituted or substituted propionamide.

The invention further provides a composition for inhibiting the telomerase activity, which includes an effective dose of anthraquinone-based compound in claim 1 and a pharmaceutical acceptable adjuvant.

Preferably, the substituent is Cl, glycine methyl ester, alanine methyl ester, valine methyl ester, leucine methyl ester, sarcosine methyl ester, glutamic acid dimethyl ester, phenylglycin methyl ester or phenylalanine methyl ester.

### BRIEF DESCRIPTION OF THE DRAWINGS

FIG. 1 shows the synthesis of 1,4-diaminoanthraquinone derivatives.

FIG. 2 shows the synthesis of 1,5-diaminoanthraquinone derivatives.

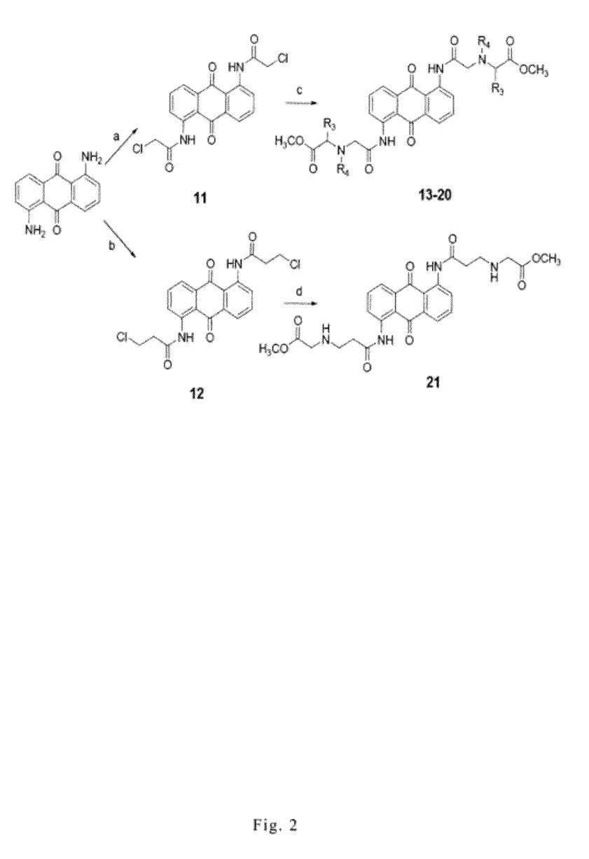

FIG. 3 shows the synthesis of 2,6-diaminoanthraquinone derivatives.

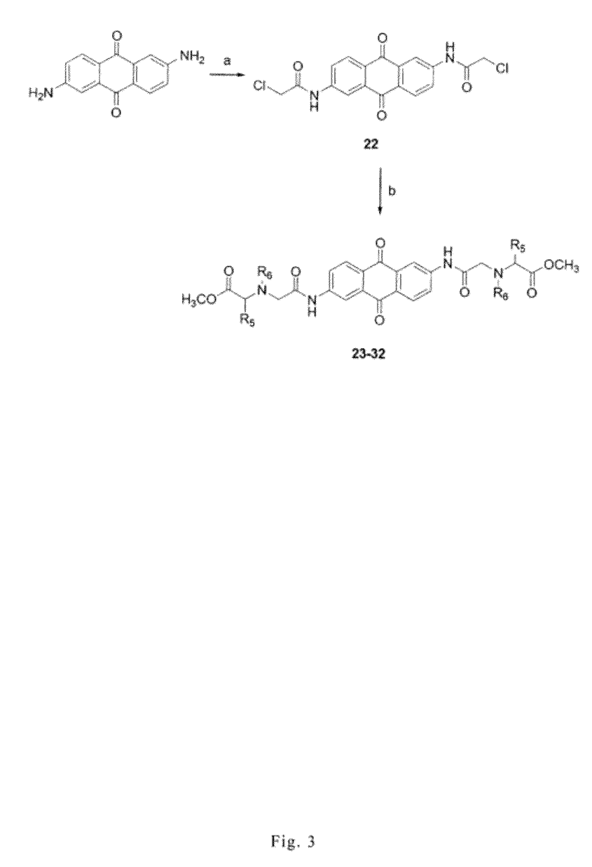

FIG. 4 shows the synthesis of 2,7-diaminoanthraquinone derivatives.

FIG. 5 shows the result of TRAP analysis.

### DETAILED DESCRIPTION OF THE INVENTION

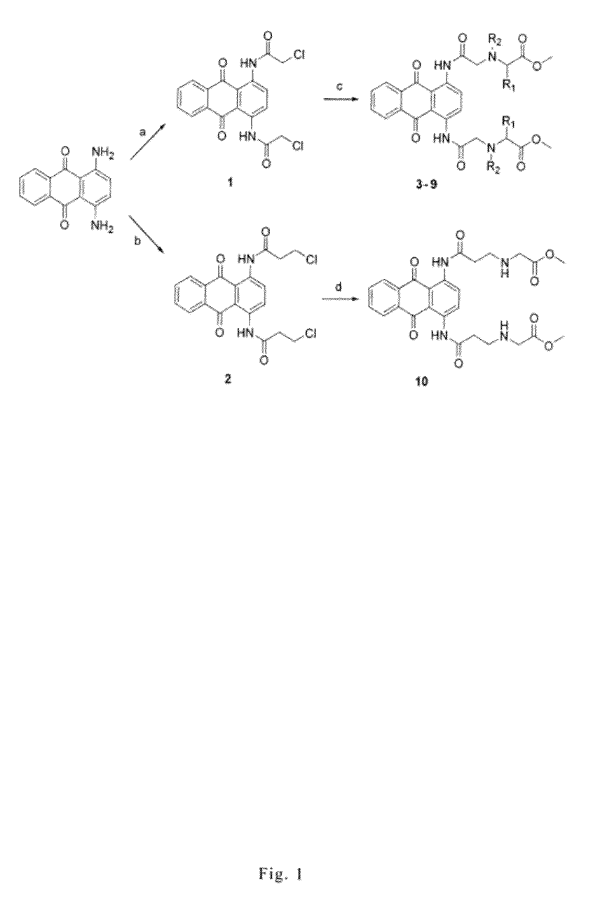

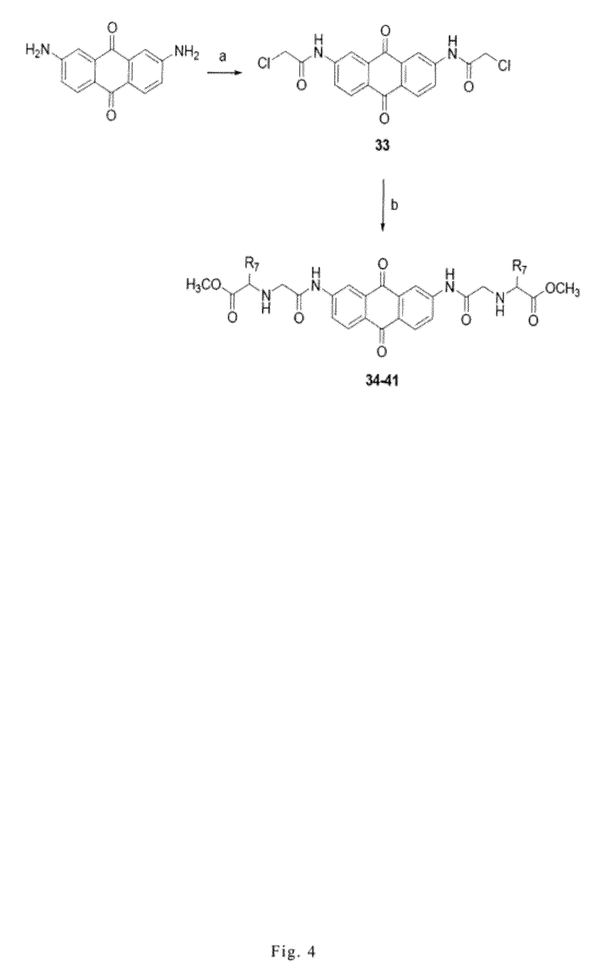

The examples are substituting aminoanthraquinone by amino acid at different carbon which include at 1,4 [symbol] 1,5 [symbol] 2,6 [symbol] 2,7 carbon to synthesize different kinds of compounds. The synthesis processes of compounds are shown as FIG. 1˜FIG. 4 and Examples 1˜41. The numbers under the compounds in the figures represent the number of the compounds, and a, b, c represent the reacting agent and reaction condition.

(1) Synthesis of 1,4-Diaminoanthraquinone Derivatives

The derivatives include Compounds 1˜10 which chemical formula are shown as Table 1.

Compound 1 was obtained as yellow powder (yield 75%). mp 284-285° C. (EtOH). 1H-NMR (300 MHz, DMSO-d₆) δ (ppm) 4.28 (s, 4H), 7.82-7.86 (m, 2H), 8.31-8.34 (m, 2H), 9.17 (s, 2H), 13.25 (s, 2H).

[Reacting Agent and Reaction Condition]:

(a) chloroacetyl chloride and pyridine reacting under room temperature for 24 hours, yield 75%.

(b) 3-chloroacetyl chloride and pyridine reacting under room temperature for 24 hours, yield 44%.

(c) and (d) both are L-amino-acid, D-amino-acid, N,N-Diisopropylethylamine (DIPEA) and Dimethylformamide (DMF).

[Synthesis Process]:
i. 1,4-diaminoanthraquinone (0.238 g, 1 mmol) was dissolved in N,N-dimethylforamide (20 mL) under an ice water bath. Added pyridine (0.5 mL) to catalyze the reaction. Next, added acyl chloride (3 mmol), removed the ice water bath under nitrogen, protected from light, and stirred for 1 day at room temperature. The reaction mixture was poured into an ice water to precipitate out the crude product. The resulting precipitate was collected by filtration, washed with hot alchol, and obtained Compound 1 and Compound 2.
ii. Glycine methyl ester hydrochloride, alanine methyl ester hydrochloride, valine methyl ester hydrochloride, leucine methyl ester hydrochloride, sarcosine methyl ester hydrochloride, glutamic acid dimethyl ester hydrochloride, phenylglycin methyl ester hydrochloride or phenylalanine methyl ester hydrochloride (3 mmol) were dissolved in anhydrous N,N′-dimethylformamide (DMF) (20 ml) respectively. DIPEA (1 ml, 6 mmol) was added to form a mixture. The mixture was then stirred in MiniClave—the compact autoclave (Buechiglasuster 0801e) for 10 min. Compound 1 and Compound 2 (0.75 mmole) were added into the mixture for 90 min under 130° C. Ethyl acetate (EA) was added to precipitate out the crude product in an ice water. The resulting precipitate was collected by filtration and purified by crystallization from n-hexane/EA to obtain Compounds 3˜10.

(2) Synthesis of 1,5-Diaminoanthraquinone Derivatives

The derivatives include Compounds 11˜21 which chemical formula are shown as Table 2.

[Reacting Agent and Reaction Condition]:

(a) chloroacetyl chloride and pyridine reacting under room temperature for 24 hours, yield 80%.

(b) 3-chloroacetyl chloride and pyridine reacting under room temperature for 24 hours, yield 45%.

(c) and (d) both are L-amino-acid, D-amino-acid, N,N-Diisopropylethylamine (DIPEA) and Dimethylformamide (DMF) putted in MiniClave—the compact autoclave (Buechiglasuster 0801e) under 130-150° C.

[Synthesis Process]:
i. 1,5-diaminoanthraquinone (0.238 g, 1 mmol) was dissolved in N,N-dimethylforamide (20 mL) under an ice water bath. Added pyridine (0.5 mL) to catalyze the reaction. Next, added acyl chloride (3 mmol), removed ice water bath under nitrogen, protected from light, and stirred for 1 day at room temperature. The reaction mixture was poured into an ice water to precipitate out the crude product. The resulting precipitate was collected by filtration, washed with hot alchol, and obtained Compound 11 and Compound 12.
ii. Glycine methyl ester hydrochloride, alanine methyl ester hydrochloride, valine methyl ester hydrochloride, leucine methyl ester hydrochloride, sarcosine methyl ester hydrochloride, glutamic acid dimethyl ester hydrochloride, phenylglycin methyl ester hydrochloride or phenylalanine methyl ester hydrochloride (3 mmol) were dissolved in anhydrous N,N′-dimethylformamide (DMF) (20 ml) respectively. DIPEA (1 ml, 6 mmol) was added to form a mixture. The mixture was then stirred in MiniClave—the compact autoclave (Buechiglasuster 0801e) for 10 min. Compound 11 and Compound 12 (0.75 mmole) were added into the mixture for 90 min under 130° C. Ethyl acetate (EA) was added to precipitate out the crude product in an ice water. The resulting precipitate was collected by filtration and purified by crystallization from n-hexane/EA to obtain Compounds 13˜21.

(3) Synthesis of 2,6-Diaminoanthraquinone Derivatives

The derivatives include Compounds 2232 which chemical formula are shown as Table 3.

[Reacting Agent and Reaction Condition]:

(a) chloroacetyl chloride and pyridine reacting under room temperature for 24 hours, yield 80%.

(b) L-amino-acid, D-amino-acid, N,N-Diisopropylethylamine (DIPEA) and Dimethylformamide (DMF) were putted in MiniClave—the compact autoclave (Buechiglasuster 0801e) under 130-150° C.

[Synthesis Process]:
i. 2,6-diaminoanthraquinone (0.238 g, 1 mmol) was dissolved in N,N-dimethylforamide (20 mL) under an ice water bath. Added pyridine (0.5 mL) to catalyze the reaction. Next, added acyl chloride (3 mmol), removed the ice water bath under nitrogen, protected from light, and stirred for 1 day at room temperature. The reaction mixture was poured into an ice water to precipitate out the crude product. The resulting precipitate was collected by filtration, washed with hot alchol, and obtained Compound 22.
ii. Glycine methyl ester hydrochloride, alanine methyl ester hydrochloride, valine methyl ester hydrochloride, leucine methyl ester hydrochloride, sarcosine methyl ester hydrochloride, glutamic acid dimethyl ester hydrochloride, phenylglycin methyl ester hydrochloride or phenylalanine methyl ester hydrochloride (3 mmol) were dissolved in anhydrous N,N′-dimethylformamide (DMF) (20 ml) respectively. DIPEA (1 ml, 6 mmol) was added to form a mixture. The mixture was then stirred in MiniClave—the compact autoclave (Buechiglasuster 0801e) for 10 min. Compound 22 (0.3 g, 0.75 mmole) was added into the mixture for 90 min under 130° C. Ethyl acetate (EA) was added to precipitate out the crude product in an ice water. The resulting precipitate was collected by filtration and purified by crystallization from n-hexane/EA to obtain Compounds 23˜32.

(4) Synthesis of 2,7-Diaminoanthraquinone Derivatives

The derivatives include Compounds 3341 which chemical formula are shown as Table 4.

[Reacting Agent and Reaction Condition]:

(a) chloroacetyl chloride and pyridine reacting under room temperature for 24 hours, yield 80%.

(b) L-amino-acid, D-amino-acid, N,N-Diisopropylethylamine (DIPEA) and Dimethylformamide (DMF) were putted in MiniClave—the compact autoclave (Buechiglasuster 0801e) under 130-150° C.

[Synthesis Process]:
i. 2,7-diaminoanthraquinone (0.238 g, 1 mmol) was dissolved in N,N-dimethylforamide (20 mL) under an ice water bath. Added pyridine (0.5 mL) to catalyze the reaction. Next, added acyl chloride (3 mmol), removed ice water bath under nitrogen, protected from light, and stirred for 1 day at room temperature. The reaction mixture was poured into an ice water to precipitate out the crude product. The resulting precipitate was collected by filtration, washed with hot alchol, and obtained Compound 33.
ii. Glycine methyl ester hydrochloride, alanine methyl ester hydrochloride, valine methyl ester hydrochloride, leucine methyl ester hydrochloride, sarcosine methyl ester hydrochloride, glutamic acid dimethyl ester hydrochloride, phenylglycin methyl ester hydrochloride or phenylalanine methyl ester hydrochloride (3 mmol) were dissolved in anhydrous N,N′-dimethylformamide (DMF) (20 ml) respectively. DIPEA (1 ml, 6 mmol) was added to form a mixture. The mixture was then stirred in MiniClave—the compact autoclave (Buechiglasuster 0801e) for 10 min. Compound 33 (0.3 g, 0.75 mmole) was added into the mixture for 90 min under 130° C. Ethyl acetate (EA) was added to precipitate out the crude product in an ice water. The resulting precipitate was collected by filtration and purified by crystallization from n-hexane/EA to obtain Compounds 34˜41.

### EXAMPLES

### Example 1

### [1,4-Bis(chloroacetamido)anthraquinone] (Compound 1)

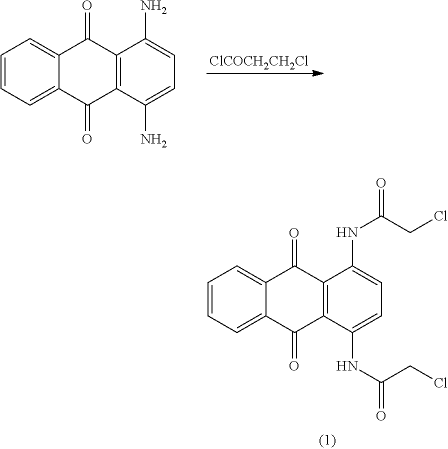

[Synthesis Process]:

1,4-diaminoanthraquinone (0.476 g, 2 mmol) was dissolved in N,N-dimethylforamide (20 ml) and pyridine (0.5 ml) under an ice water bath. Added chloroacetyl chloride (0.5 ml, 6 mmol), removed the ice water bath under nitrogen, protected from light, and stirred for 1 day at room temperature. The reaction mixture was poured into a small amount of crushed ice to precipitate out the crude product. The resulting precipitate was collected by filtration, washed with diethyl ether, and purified by crystallization from ethanol. Compound 1 was obtained (yield 75%). mp 284-285° C. (EtOH). 1H-NMR (300 MHz, DMSO-d6) δ (ppm) 4.28 (s, 4H), 7.82-7.86 (m, 2H), 8.31-8.34 (m, 2H), 9.17 (s, 2H), 13.25 (s, 2H).

### Example 2

### [1,4-Bis(3-chloropropionamido)anthraquinone] (Compound 2)

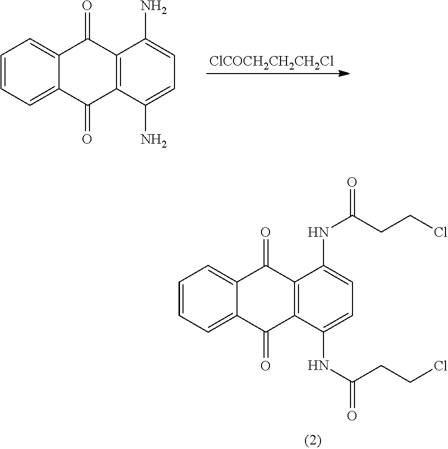

[Synthesis Process]:

1,4-diaminoanthraquinone (0.476 g, 2 mmol) was dissolved in N,N-dimethylforamide (20 ml) and pyridine (0.5 ml) under an ice water bath. Added chloroacetyl chloride (0.5 ml, 6 mmol), removed the ice water bath under nitrogen, protected from light, and stirred for 1 day at room temperature. The reaction mixture was poured into a small amount of crushed ice to precipitate out the crude product. The resulting precipitate was collected by filtration, washed with diethyl ether, and purified by crystallization from ethanol. Compound 2 was obtained as yellow powder (yield 44%). mp 225-226° C. (EtOH). 1H-NMR (300 MHz, CDCl₃) δ (ppm) 3.01 (t, J=6.3 Hz, 4H), 3.93 (t, J=6.6 Hz, 4H), 7.82-7.85 (m, 2H), 8.26-8.29 (m, 2H), 9.17 (s, 2H), 12.26 (s, 2H).

### Example 3

### [1,4-Bis[2-(glycin methyl ester)acetamido]anthraquinone] (Compound 3)

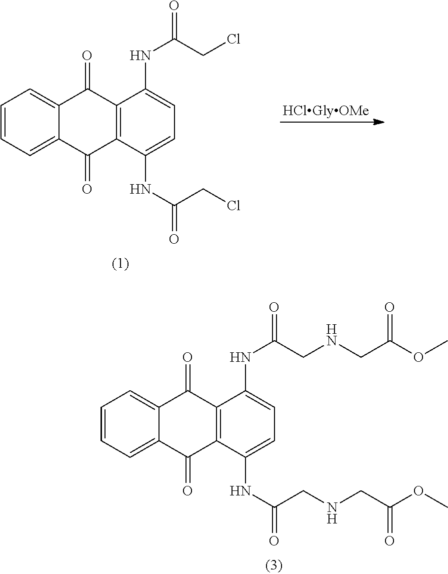

[Synthesis Process]:

Compound 1 (0.5 mmol) was dissolved in N,N-dimethylformamide (20 ml) and DIPEA (1 mL, 6 mmole) and glycine methyl ester hydrochloride (0.37 g, 3 mmole) were added under nitrogen. The mixture was stirred for 48 h at room temperature. The reaction mixture was poured into an ice water to precipitate out the crude product. The resulting precipitate was collected by filtration, washed with diethyl ether, and purified by crystallization from ethyl acetate. Compound 3 was obtained as red brown powder (yield 30%). R_{f}: 0.22 (ethyl acetate:n-hexane=1:1). Mp: 164˜165° C. (EtOH). 1H-NMR (300 MHz, CDCl₃) δ (ppm) 3.59 (s, 4H), 3.63 (s, 4H), 3.79 (s, 6H), 7.80 (t, J=6.8 Hz, 2H), 8.29 (t, J=6.6 Hz, 2H), 9.22 (s, 2H). 13C-NMR (75 MHz, CDCl₃) δ ppm 50.63, 51.98, 53.51, 117.71, 127.00, 128.82, 133.21, 134.20, 137.47, 171.58 (NCO), 172.52 (CCO), 186.35 (CO). HRMS (ESI) m/z calcd for C₂₄H₂₄N₄O₈ [M+H]⁺: 497.1594. Found: 497.1657.

### Example 4

### [(L)-1,4-Bis[2-(alanine methyl ester) acetamido]anthraquinone] (Compound 4)

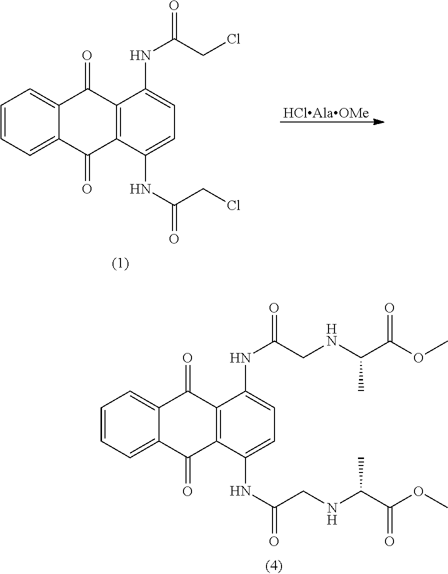

[Synthesis Process]:

Compound 1 (0.5 mmol) was dissolved in N,N-dimethylformamide (20 ml) and DIPEA (1 mL, 6 mmole) and L-alanine methyl ester hydrochloride (0.42 g, 3 mmole) were added under nitrogen. The mixture was stirred in mini-reactor for 90 min at 130-150° C. The reaction mixture was poured into an ice water to precipitate out the crude product. The resulting precipitate was then extracted with ethyl acetate, and purified by crystallization from ethyl acetate/n-hexane. Compound 4 was obtained as red brown powder (yield 26%). R_{f}: 0.23 (ethyl acetate:n-hexane=1:1). mp: 149-150° C. (EtOH). 1H-NMR (300 MHz, CDCl₃) δ ppm 1.56 (d, J=6.9 Hz 6H), 3.33 (d, J=17.4 Hz, 2H), 3.70 (d, J=17.7 Hz 2H), 3.47-3.54 (m, 2H), 3.76 (s, 6H), 7.78-7.80 (m, 2H), 8.26-8.29 (m, 2H), 9.22 (s, 2H), 13.25 (s, 2H). 13C-NMR (75 MHz, CDCl₃) δ ppm 19.00, 46.16, 52.14, 56.87, 117.76, 126.93, 128.75, 133.25, 134.15, 137.36, 171.89 (NCO), 175.76 (CCO), 186.22 (CO). HRMS (ESI) m/z calcd for C₂₆H₂₈N₄O₈ [M+H]⁺: 525.1907. Found: 525.1974.

### Example 5

### [(D)-1,4-Bis[2-(alanine methyl ester) acetamido]anthraquinone L)](Compound 5)

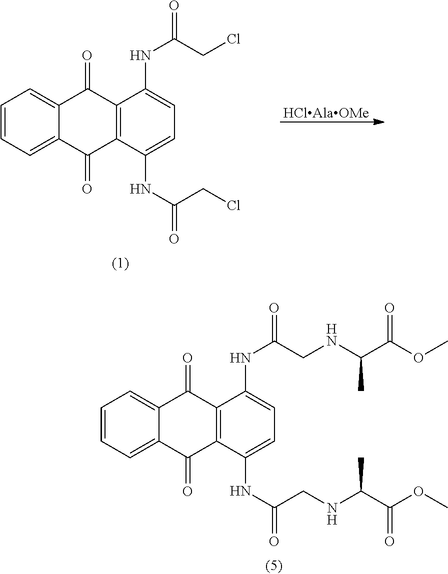

[Synthesis Process]:

DIPEA (1 ml, 6 mmole) was added in (D) alanine methyl ester hydrochloride (0.42 g, 3 mmole) and the mixture was dissolved in N,N-dimethylforamide (20 mL). Next, Compound 1 (0.196 g, 0.5 mmol) was added. The mixture was then stirred in MiniClave—the compact autoclave (Buechiglasuster 0801e) for 90 min under the oil bath at 130˜150° C. Ethyl acetate (EA) was added to precipitate out the crude product in an ice water. The resulting precipitate was collected by filtration and purified by crystallization from n-hexane/EA to obtain Compound 5.

Compound 5 was obtained as red brown powder (yield 33%). R_{f}: 0.23 (ethyl acetate:n-hexane=1:1). mp: 149˜150° C. (EtOH). 1H-NMR (300 MHz, CDCl₃) δ ppm 1.56 (d, J=6.9 Hz, 6H), 3.32 (d, J=17.4 Hz, 2H), 3.70 (d, J=17.7 Hz, 2H), 3.46-3.53 (m, 2H), 3.77 (s, 6H), 7.78-7.80 (m, 2H), 8.26-8.29 (m, 2H), 9.22 (s, 2H), 13.26 (s, 2H). 13C-NMR (75 MHz, CDCl₃) δ ppm 18.99, 46.89, 52.12, 56.86, 117.68, 126.89, 128.69, 133.20, 134.12, 137.40, 171.94 (NCO), 175.74 (CCO), 186.15 (CO). HRMS (ESI) m/z calcd for C₂₆H₂₈N₄O₈ [M+H]⁺: 525.1907. Found: 525.1967.

### Example 6

### [(L)-1,4-Bis[2-(valine methyl ester) acetamido]anthraquinone] (Compound 6)

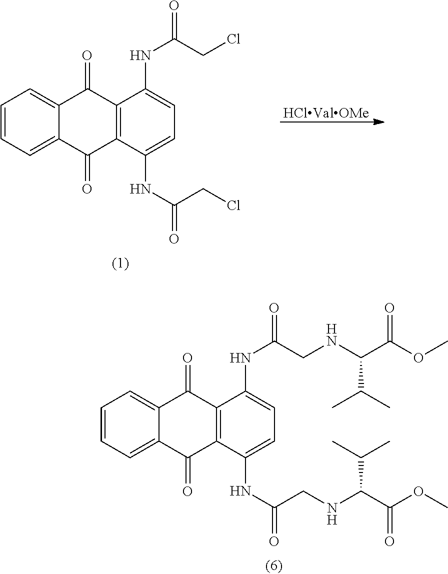

[Synthesis Process]:

DIPEA (1 ml, 6 mmole) was added in (L)-valine methyl ester hydrochloride] (0.50 g, 3 mmole) and the mixture was dissolved in N,N-dimethylforamide (20 mL). Next, Compound 1 (0.196 g, 0.5 mmol) was added. The mixture was then stirred in MiniClave—the compact autoclave (Buechiglasuster 0801e) for 90 min under the oil bath at 130˜150° C. Ethyl acetate (EA) was added to precipitate out the crude product in an ice water. The resulting precipitate was collected by filtration and purified by crystallization from n-hexane/EA to obtain Compound 6.

Compound 6 was obtained as red brown powder (yield 30%). R_{f}: 0.36 (ethyl acetate:n-hexane=1:1). mp 180˜181° C. (EtOH). 1H-NMR (300 MHz, CDCl₃) δ ppm 1.08-1.15 (m, 12H), 2.15-2.21 (m, 2H), 3.17 (d, J=5.1 Hz, 2H), 3.24 (d, J=7.4 Hz, 2H), 3.70 (d, J=17.1 Hz, 2H), 3.76 (s, 6H), 7.77-7.80 (m, 2H), 8.21-8.24 (m, 2H), 9.17 (s, 2H), 13.09 (s, 2H). 13C-NMR (75 MHz, CDCl₃) δ ppm 18.53, 19.21, 31.73, 51.73, 53.18, 67.83, 118.15, 126.94, 128.97, 133.41, 134.18, 137.43, 171.77 (NCO), 174.86 (CCO), 186.22 (CO). HRMS (ESI) m/z calcd for C₃₀H₃₆N₄O₈ [M+H]⁺: 581.2533. Found: 581.2603.

### Example 7

### [(D)-1,4-Bis[2-(valine methyl ester)acetamido]anthraquinone] (Compound 7)

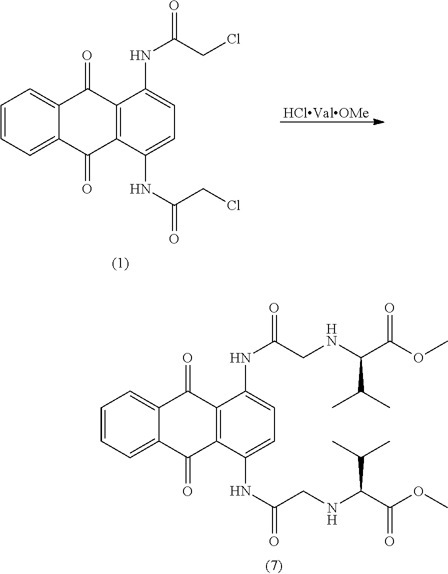

[Synthesis Process]:

DIPEA (1 ml, 6 mmole) was added in (D) valine methyl ester hydrochloride (0.50 g, 3 mmole) and the mixture was dissolved in N,N-dimethylforamide (20 mL). Next, Compound 1 (0.196 g, 0.5 mmol) was added. The mixture was then stirred in MiniClave—the compact autoclave (Buechiglasuster 0801e) for 90 min under the oil bath at 130˜150° C. Ethyl acetate (EA) was added to precipitate out the crude product in an ice water. The resulting precipitate was collected by filtration and purified by crystallization from n-hexane/EA to obtain Compound 7.

Compound 7 was obtained as red brown powder (yield 43%). R_{f}: 0.36 (ethyl acetate:n-hexane=1:1). mp 180˜181° C. (EtOH). 1H-NMR (300 MHz, CDCl₃) δ ppm 1.10-1.17 (m, 12H), 2.22-2.28 (m, 2H), 3.27 (d, J=5.4 Hz, 2H), 3.36 (d, J=17.1 Hz, 2H), 3.71 (t, J=7.8 Hz, 2H), 3.78 (s, 6H), 7.78-7.80 (m, 2H), 8.21-8.24 (m, 2H), 9.15 (s, 2H), 13.07 (s, 2H). 13C-NMR (75 MHz, CDCl₃) δ ppm 18.50, 19.16, 31.68, 51.66, 53.12, 67.79, 117.95, 126.85, 128.79, 133.28, 134.10, 137.34, 171.70 (NCO), 174.80 (CCO), 186.03 (CO). HRMS (ESI) m/z calcd for C₃₀H₃₆N₄O₈ [M+H]⁺: 581.2533. Found: 581.2596.

### Example 8

### [(L)-1,4-Bis[2-(leucine methyl ester) acetamido]anthraquinone] (Compound 8)

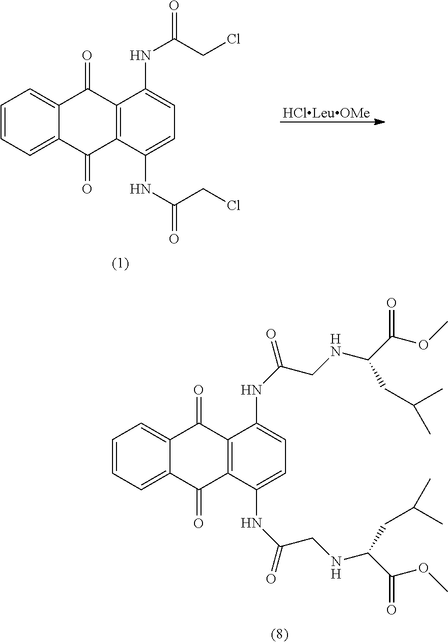

[Synthesis Process]:

DIPEA (1 ml, 6 mmole) was added in (L) leucine methyl ester hydrochloride] (0.55 g, 3 mmole) and the mixture was dissolved in N,N-dimethylforamide (20 mL). Next, Compound 1 (0.196 g, 0.5 mmol) was added. The mixture was then stirred in MiniClave—the compact autoclave (Buechiglasuster 0801e) for 90 min under the oil bath at 130˜150° C. Ethyl acetate (EA) was added to precipitate out the crude product in an ice water. The resulting precipitate was collected by filtration and purified by crystallization from n-hexane/EA to obtain Compound 8.

Compound 8 was obtained as red brown powder (yield 39%). R_{f}: 0.34 (ethyl acetate:n-hexane=1:1). mp 200-201° C. (EtOH). 1H-NMR (300 MHz, CDCl₃) δ ppm 0.92-0.98 (m, 12H), 1.58-2.03 (m, 6H), 3.27 (d, J=17.1 Hz, 2H), 3.69 (d, J=17.4 Hz, 2H), 3.41 (t, 2H), 3.75 (s, 6H), 7.79-7.82 (m, 2H), 8.23-8.26 (m, 2H), 9.21 (s, 2H), 13.16 (s, 2H). 13C-NMR (75 MHz, CDCl₃) δ ppm 22.43, 22.79, 24.65, 42.66, 51.87, 52.46, 60.26, 117.76, 126.82, 128.81, 133.38, 134.15, 137.44, 171.81 (NCO), 175.79 (CCO), 186.11 (CO). HRMS (ESI) m/z calcd for C₃₂H₄₀N₄O₈ [M+H]⁺: 609.2846. Found: 609.2909.

### Example 9

### [1,4-Bis[2-(sarcosine methyl ester)acetamido]anthraquinone] (Compound 9)

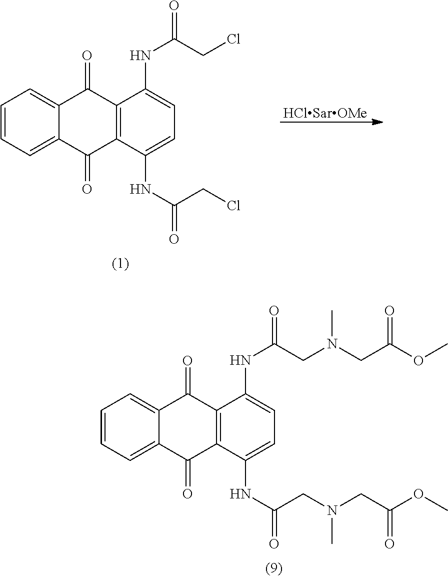

[Synthesis Process]:

DIPEA (1 ml, 6 mmole) was added in sarcosine methyl ester hydrochloride (0.42 g, 3 mmole) and the mixture was dissolved in N,N-dimethylforamide (20 mL). Next, Compound 1 (0.196 g, 0.5 mmol) was added. The mixture was then stirred in MiniClave—the compact autoclave (Buechiglasuster 0801e) for 90 min under the oil bath at 130˜150° C. Ethyl acetate (EA) was added to precipitate out the crude product in an ice water. The resulting precipitate was collected by filtration and purified by crystallization from n-hexane/EA to obtain Compound 9.

Compound 9 was obtained as red brown powder (yield 45%). R_{f}: 0.43 (ethyl acetate:n-hexane=1:1). mp 150˜151° C. (EtOH). 1H-NMR (300 MHz, CDCl₃) 5 ppm 2.67 (s, 6H), 3.53 (s, 4H), 3.64 (s, 4H), 3.75 (s, 6H), 7.77-7.80 (m, 2H), 8.27-8.30 (m, 2H), 9.20 (s, 2H), 13.21 (s, 2H). 13C-NMR (75 MHz, CDCl₃) δ ppm 42.85, 51.51, 57.95, 61.31, 118.00, 126.99, 128.77, 133.30, 134.02, 137.36, 171.07 (NCO), 171.32 (CCO), 186.21 (CO). ESI-MS m/z: 525.3 [M+H]⁺.

### Example 10

### [1,4-Bis[2-(glycin methyl ester) propionamido]anthraquinone] (Compound 10)

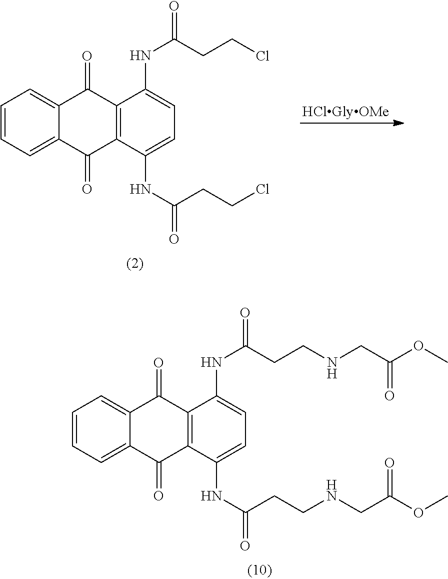

[Synthesis Process]:

DIPEA (1 ml, 6 mmole) was added in glycine methyl ester hydrochloride (0.37 g, 3 mmole) and the mixture was dissolved in N,N-dimethylforamide (20 mL). Next, Compound 2 (0.196 g, 0.5 mmol) was added. The mixture was then stirred in MiniClave—the compact autoclave (Buechiglasuster 0801e) for 90 min under the oil bath at 130˜150° C. Ethyl acetate (EA) was added to precipitate out the crude product in an ice water. The resulting precipitate was collected by filtration and purified by crystallization from n-hexane/EA to obtain Compound 10.

Compound 10 was obtained as red brown powder (yield 23%). R_{f}: 0.22 (ethyl acetate:n-hexane=1:1). mp 155˜156° C. (EtOH). 1H-NMR (300 MHz, CDCl₃) δ ppm 2.50 (s, 2H), 2.73 (t, 4H, —CH₂—), 3.07 (t, 4H, —CH₂—), 3.51 (s, 4H, —CH₂—), 3.73 (s, 6H, —OCH₃—), 7.79-7.82 (m, 2H), 8.24-8.67 (m, 2H), 9.13 (s, 2H) 12.59 (s, 2H). 13C-NMR (75 MHz, CDCl₃) δ ppm 38.91, 45.13, 50.76, 51.82, 116.98, 127.09, 129.21, 133.32, 134.43, 138.26, 171.57 (NCO), 172.73 (CCO), 186.85 (CO). HRMS (ESI) m/z calcd for C₂₆H₂₈N₄O₈ [M+H]⁺: 525.1907. Found: 525.1960.

### Example 11

### [1,5-Bis(chloroacetamido)anthraquinone] (Compound 11)

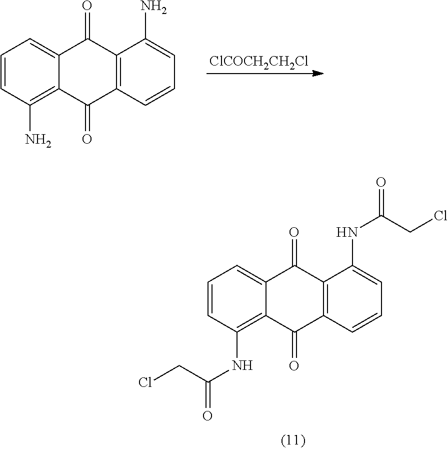

[Synthesis Process]:

1,5-diaminoanthraquinone (0.476 g, 2 mmol) was dissolved in DIPEA (20 ml) and pyridine (0.5 ml) under an ice water bath. Added chloroacetyl chloride (0.5 ml, 6 mmol), removed the ice water bath under nitrogen, protected from light, and stirred for 1 day at room temperature. The reaction mixture was poured into an ice water to precipitate out the crude product. The resulting precipitate was collected by filtration, washed with diethyl ether, and purified by crystallization from ethanol. Compound 11 was obtained. Yield: 80%. mp: 349-350° C. (EtOH) (lit31 mp 370° C.). 1H-NMR (300 MHz, DMSO-d6) δ (ppm) 4.35 (s, 4H, —CH2-), 7.82 (t, J=8.1 Hz 2H, H-3,7), 8.17 (d, J=7.2 Hz 2H, H-4,8), 9.14 (d, J=8.7 Hz 2H, H-2,6), 11.90 (s, 2H, Ar—NH—).

### Example 12

### [(3-chloropropionamido)anthraquinone] (Compound 12)

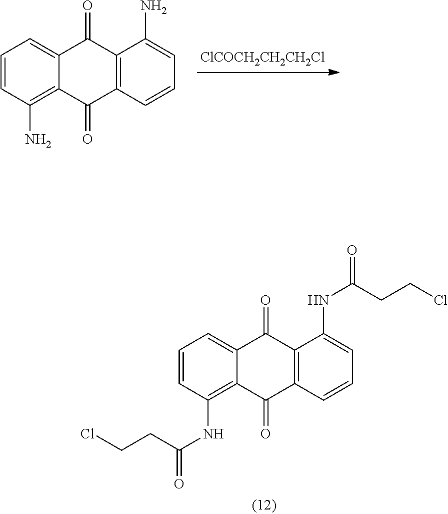

[Synthesis Process]:

1,5-diaminoanthraquinone (0.476 g, 2 mmol) was dissolved in N,N-dimethylforamide (20 ml) and pyridine (0.5 ml) under an ice water bath. Added 3-chloroacetyl chloride (0.5 ml, 6 mmol), removed the ice water bath under nitrogen, protected from light, and stirred for 1 day at room temperature. The reaction mixture was poured into an ice water to precipitate out the crude product. The resulting precipitate was collected by filtration, washed with diethyl ether, and purified by crystallization from ethanol. Compound 12 was obtained.

Yield: 45%. mp 275-276° C. (EtOH) (lit31 mp 275° C.) 1H-NMR (300 MHz, CDCl3) δ (ppm): 3.03 (t, J=6.6 Hz, 4H, —COCH₂—), 3.94 (t, J=6.6 Hz, 4H, —CH2Cl), 7.81 (t, J=8.1 Hz 2H, H-3,7), 8.08 (d, 2H, H-4,8), 9.16 (d, J=8.7 Hz 2H, H-2,6), 12.41 (s, 2H, Ar—NH—).

### Example 13

### [(L)-1,5-Bis[2-alanine methyl ester) acetamido]anthraquinone] (Compound 13)

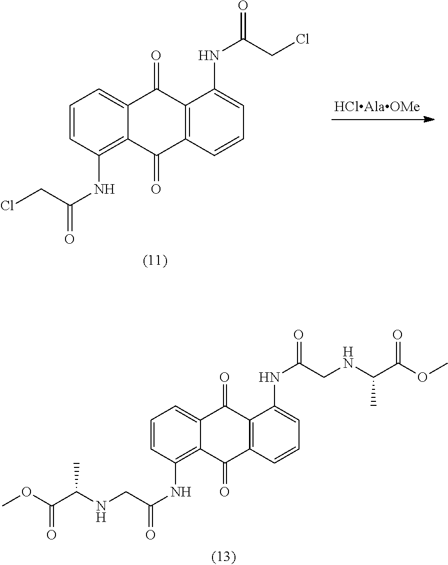

[Synthesis Process]:

DIPEA (1 ml, 6 mmole) was added in (L) alanine methyl ester hydrochloride (0.42 g, 3 mmole) and the mixture was dissolved in N,N-dimethylforamide (20 mL). Next, Compound 11 (0.196 g, 0.5 mmol) was added. The mixture was then stirred in MiniClave—the compact autoclave (Buechiglasuster 0801e) for 90 min under the oil bath at 130˜150° C. Ethyl acetate (EA) was added to precipitate out the crude product in an ice water. The resulting precipitate was collected by filtration and purified by crystallization from n-hexane/EA to obtain Compound 13.

Compound 13 was obtained as brown powder (yield 40%). R_{f}: 0.26 (ethyl acetate:n-hexane=2:1). mp 141-142° C. (EtOH). 1H-NMR (300 MHz, CDCl₃) δ ppm 1.56 (d, J=7.2 Hz 6H), 3.32 (d, J=17.7 Hz, 2H), 3.70 (d, J=17.4 Hz, 2H), 3.50 (dd, J=15.0, 7.2 Hz, 2H), 3.76 (s, 611), 7.75 (t, J=8.1 Hz, 2H), 8.05 (d, J=7.5 Hz, 211), 9.20 (d, J=8.7 Hz, 2H), 13.09 (s, 2H). 13C-NMR (75 MHz, CDCl₃) δ ppm 18.99, 52.01, 52.05, 56.89, 117.60, 122.60, 125.95, 134.53, 135.44, 140.76, 172.20 (NCO), 175.73 (CCO), 185.77 (CO). ESI-MS m/z: 525.2 [M+H]⁺

### Example 14

### [(D)-1,5-Bis[2-(alanine methyl ester) acetamido]anthraquinone] (Compound 14)

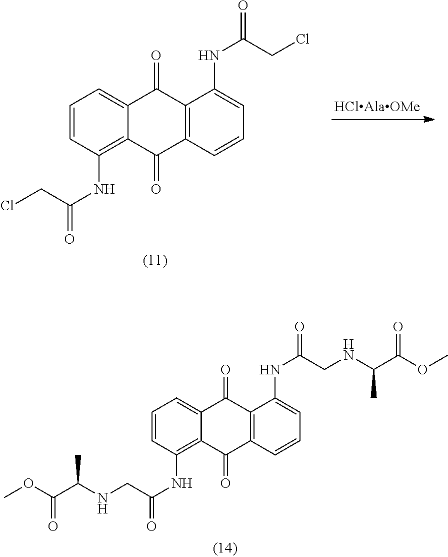

[Synthesis Process]:

DIPEA (1 ml, 6 mmole) was added in (D) alanine methyl ester hydrochloride (0.42 g, 3 mmole) and the mixture was dissolved in N,N-dimethylforamide (20 mL). Next, Compound 11 (0.196 g, 0.5 mmol) was added. The mixture was then stirred in MiniClave—the compact autoclave (Buechiglasuster 0801e) for 90 min under the oil bath at 130-150° C. Ethyl acetate (EA) was added to precipitate out the crude product in an ice water. The resulting precipitate was collected by filtration and purified by crystallization from n-hexane/EA to obtain Compound 14.

Compound 14 was obtained as yellow powder (yield 45%). R_{f}: 0.26 (ethyl acetate:n-hexane=2:1). mp 143˜144° C. (EtOH). 1H-NMR (300 MHz, CDCl₃) δ ppm 1.57 (d, J=6.9 Hz, 6H), 3.32 (d, J=17.7 Hz, 2H), 3.71 (d, J=17.4 Hz, 2H), 3.52 (dd, J=14.1, 7.2 Hz, 2H), 3.77 (s, 6H, —OCH₃), 7.75 (t, J=9.0 Hz, 2H), 8.05 (d, J=7.5 Hz, 2H), 9.20 (d, J=8.4 Hz, 2H), 13.10 (s, 2H). 13C-NMR (75 MHz, CDCl₃) δ ppm 18.99, 52.01, 52.05, 56.89, 117.61, 122.60, 125.95, 134.54, 135.44, 140.76, 172.18 (NCO), 175.72 (CCO), 185.77 (CO). ESI-MS m/z: 525.2 [M+H]⁺.

### Example 15

### [L-1,5-Bis[2-(valine methyl ester)acetamido]anthraquinone] (Compound 15)

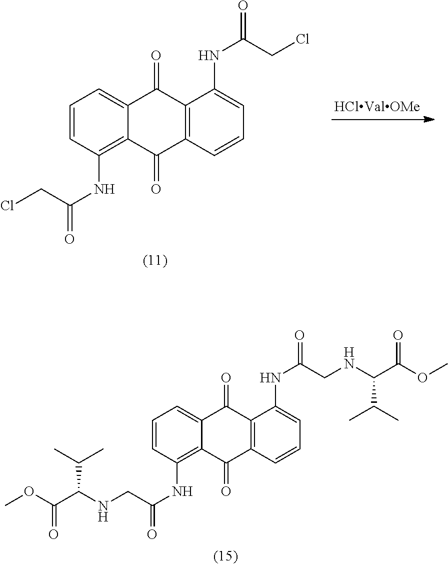

[Synthesis Process]:

DIPEA (1 ml, 6 mmole) was added in (L) valine methyl ester hydrochloride (0.50 g, 3 mmole) and the mixture was dissolved in N,N-dimethylforamide (20 mL). Next, Compound 11 (0.196 g, 0.5 mmol) was added. The mixture was then stirred in MiniClave—the compact autoclave (Buechiglasuster 0801e) for 90 min under the oil bath at 130˜150° C. Ethyl acetate (EA) was added to precipitate out the crude product in an ice water. The resulting precipitate was collected by filtration and purified by crystallization from n-hexane/EA to obtain Compound 15.

Compound 15 was obtained as yellow brown powder (yield 35%). R_{f}: 0.45 (ethyl acetate:n-hexane=2:1). mp 125-126° C. (EtOH). 1H-NMR (300 MHz, CDCl₃) δ ppm 1.09-1.16 (m, 12H, —CH₃), 2.19-2.23 (m, 2H, —CH—), 3.22 (d, J=5.1 Hz, 2H, —CH—), 3.31 (d, J=16.8 Hz, 2H, —CH₂—), 3.74 (d, J=18.5 Hz, 2H, —CH₂—), 3.77 (s, 6H, —OCH₃), 7.75 (t, J=8.1 Hz, 2H), 8.01 (d, J=7.8 Hz, 2H), 9.15 (d, J=8.4 Hz, 2H), 12.92 (s, 2H, Ar—NH—). 13C-NMR (75 MHz, CDCl₃) δ ppm 18.48, 19.14, 31.67, 51.68, 53.10, 67.83, 117.76, 122.57, 126.10, 134.61, 135.41, 140.72, 171.97 (NCO), 174.78 (CCO), 185.52 (CO). ESI-MS m/z: 581.3 [M+H]⁺.

### Example 16

### [(D)-1,5-Bis[2-(valine methyl ester) acetamido]anthraquinone] (Compound 16)

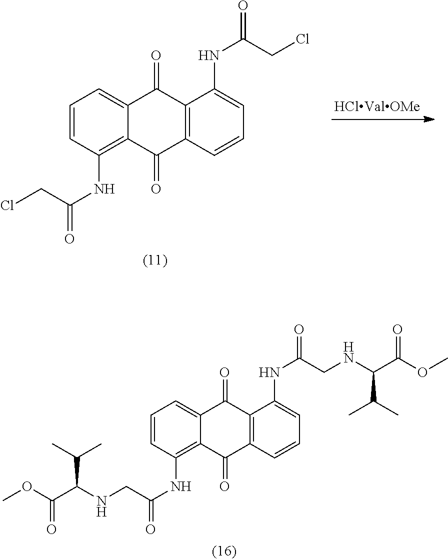

[Synthesis Process]:

DIPEA (1 ml, 6 mmole) was added in (D) valine methyl ester hydrochloride (0.50 g, 3 mmole) and the mixture was dissolved in N,N-dimethylforamide (20 mL). Next, Compound 11 (0.196 g, 0.5 mmol) was added. The mixture was then stirred in MiniClave—the compact autoclave (Buechiglasuster 0801e) for 90 min under the oil bath at 130˜150° C. Ethyl acetate (EA) was added to precipitate out the crude product in an ice water. The resulting precipitate was collected by filtration and purified by crystallization from n-hexane/EA to obtain Compound 16.

Compound 16 was obtained as yellow brown powder (yield 41%). R_{f}: 0.45 (ethyl acetate:n-hexane=2:1). mp 126-127° C. (EtOH). 1H-NMR (300 MHz, CDCl₃) δ ppm 1.08-1.19 (m, 12H, —CH₃), 2.17-2.23 (m, 2H, —CH—), 3.19 (d, J=5.1 Hz, 2H, —CH—), 3.28 (d, J=17.4 Hz, 2H, —CH₂—), 3.72 (d, J=17.4 Hz, 2H, —CH₂—), 3.76 (s, 6H, —OCH₃), 7.74 (t, J=8.1 Hz, 2H), 8.01 (d, J=7.5 Hz, 2H), 9.15 (d, J=8.4 Hz, 2H), 12.92 (s, 2H, Ar—NH—). 13C-NMR (75 MHz, CDCl₃) δ ppm 18.51, 19.17, 31.70, 51.72, 53.14, 67.85, 117.88, 122.65, 126.19, 134.71, 135.47, 140.77, 171.98 (NCO), 174.80 (CCO), 185.66 (CO). ESI-MS m/z: 581.3 [M+H]⁺.

### Example 17

### [(L)-1,5-Bis[2-(leucine methyl ester) acetamido]anthraquinone] (Compound 17)

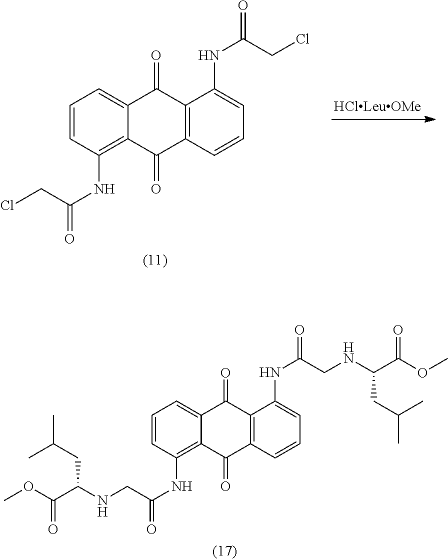

[Synthesis Process]:

DIPEA (1 ml, 6 mmole) was added in (L) leucine methyl ester hydrochloride (0.55 g, 3 mmole) and the mixture was dissolved in N,N-dimethylforamide (20 mL). Next, Compound 11 (0.196 g, 0.5 mmol) was added. The mixture was then stirred in MiniClave—the compact autoclave (Buechiglasuster 0801e) for 90 min under the oil bath at 130˜150° C. Ethyl acetate (EA) was added to precipitate out the crude product in an ice water. The resulting precipitate was collected by filtration and purified by crystallization from n-hexane/EA to obtain Compound 17.

Compound 17 was obtained as yellow brown powder (yield 35%). R_{f}: 0.47 (ethyl acetate:n-hexane=1:1). mp 149-150° C. (EtOH). 1H-NMR (300 MHz, CDCl₃) δ ppm 0.92˜0.98 (m, 12H, —CH₃), 1.62-2.04 (m, 6H, —CH₂—CH—), 3.29 (d, J=17.1 Hz, 2H, —CH₂—), 3.71 (d, J=17.4 Hz, 2H, —CH₂—), 3.42 (t, J=6.6 Hz, 2H, —CH—), 3.76 (s, 6H, —OCH₃), 7.76 (t, J=8.1 Hz, 2H), 8.01 (d, J=7.8 Hz, 2H), 9.18 (d, J=8.7 Hz, 2H), 12.97 (s, 2H, Ar—NH—). 13C-NMR (75 MHz, CDCl₃) δ ppm 22.43, 22.76, 24.63, 42.66, 46.12, 51.88, 52.42, 60.29, 117.76, 122.48, 126.07, 134.69, 135.52, 140.83, 172.02 (NCO), 175.77 (CCO), 185.64 (CO). ESI-MS m/z: 609.4 [M+H]⁺.

### Example 18

### [1,5-Bis[2-(glutamic acid dimethyl ester) acetamido]anthraquinone] (Compound 18)

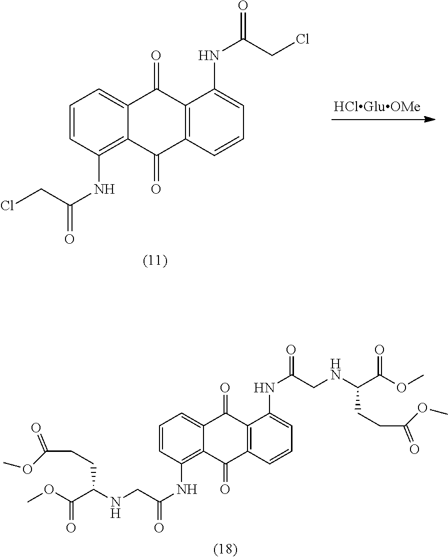

[Synthesis Process]:

DIPEA (1 ml, 6 mmole) was added in glutamic acid dimethyl ester hydrochloride (0.63 g, 3 mmole) and the mixture was dissolved in N,N-dimethylforamide (20 mL). Next, Compound 11 (0.196 g, 0.5 mmol) was added. The mixture was then stirred in MiniClave—the compact autoclave (Buechiglasuster 0801e) for 90 min under the oil bath at 130˜150° C. Ethyl acetate (EA) was added to precipitate out the crude product in an ice water. The resulting precipitate was collected by filtration and purified by crystallization from n-hexane/EA to obtain Compound 18.

Compound 18 was obtained as red brown powder (yield 38%). R_{f}: 0.32 (ethyl acetate:n-hexane=2:1). mp 151-152° C. (EtOH). 1H-NMR (300 MHz, CDCl₃) δ ppm 2.11-2.25 (m, 4H, —CH₂—), 2.71-2.86 (m, 4H, —CH₂—), 3.35 (d, J=17.4 Hz, 2H, —CH₂—), 3.65 (d, J=17.1 Hz, 2H, —CH₂—), 3.50 (t, J=6.0 Hz, 2H, —CH—), 3.62 (s, 6H, —OCH₃), 3.78 (s, 6H, —OCH₃), 7.74 (t, J=9.6 Hz, 2H), 8.03 (d, J=7.5 Hz 2H), 9.16 (d, J=8.7 Hz, 2H), 12.97 (s, 2H, Ar—NH—). 13C-NMR (75 MHz, CDCl₃) δ ppm 28.34, 30.08, 51.58, 52.15, 52.62, 60.98, 117.64, 122.74, 126.03, 134.58, 135.55, 140.78, 171.78 (NCO), 173.50 (CCO), 174.69 (CCO), 185.73 (CO). ESI-MS m/z: 669.4 [M+H]⁺.

### Example 19

### [1,5-Bis[2-(sarcosine methyl ester) acetamido]anthraquinone] (Compound 19)

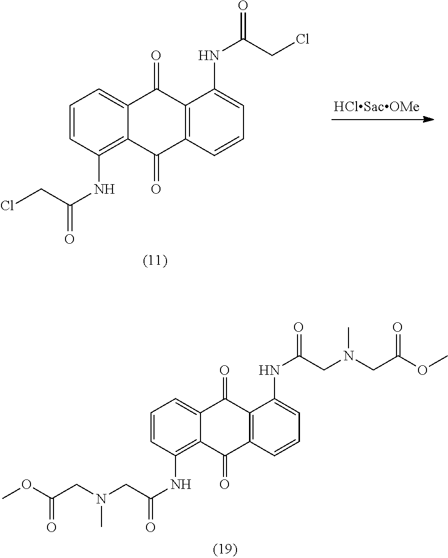

[Synthesis Process]:

DIPEA (1 ml, 6 mmole) was added in sarcosine methyl ester hydrochloride (0.42 g, 3 mmole) and the mixture was dissolved in N,N-dimethylforamide (20 mL). Next, Compound 11 (0.196 g, 0.5 mmol) was added. The mixture was then syirred in MiniClave—the compact autoclave (Buechiglasuster 0801e) for 90 min under the oil bath at 130˜50° C. Ethyl acetate (EA) was added to precipitate out the crude product in an ice water. The resulting precipitate was collected by filtration and purified by crystallization from n-hexane/EA to obtain Compound 19.

Compound 19 was obtained as red brown powder (yield 55%). R_{f}: 0.48 (ethyl acetate:n-hexane=1:1). mp 186-187° C. (EtOH). 1H-NMR (300 MHz, CDCl₃) δ ppm 2.67 (s, 6H, —N—CH₃), 3.53 (s, 4H, —CH₂—), 3.64 (s, 4H, —CH₂—), 3.75 (s, 6H, —OCH₃), 7.76 (t, J=8.4 Hz 2H), 8.07 (d, J=6.6 Hz, 2H), 9.17 (d, J=7.5 Hz, 2H), 13.05 (s, 2H, Ar—NH—). 13C-NMR (75 MHz, CDCl₃) δ ppm 22.42, 22.75, 24.63, 42.65, 51.87, 52.41, 60.29, 117.76, 122.48, 126.07, 134.68, 135.51, 140.82, 172.02 (NCO), 175.76 (CCO), 185.64 (CO). ESI-MS m/z: 525.3 [M+H]⁺.

### Example 20

### 1,5-Bis[2-(β-alanine methyl ester) acetamido]anthraquinone (Compound 20)

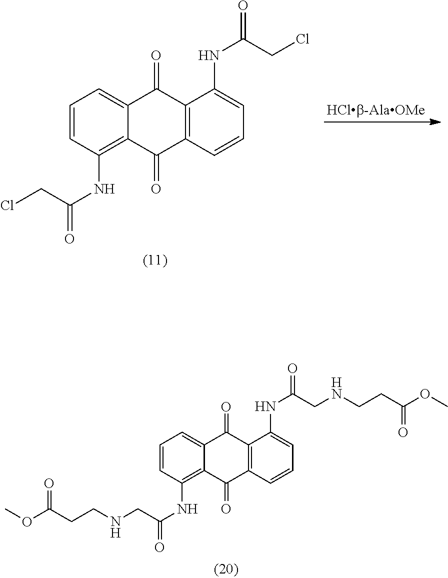

[Synthesis Process]:

DIPEA (1 ml, 6 mmole) was added in 13-alanine methyl ester hydrochloride (0.42 g, 3 mmole) and the mixture was dissolved in N,N-dimethylforamide (20 mL). Next, compound 11 (0.196 g, 0.5 mmol) was added. The mixture was then stirred in MiniClave—the compact autoclave (Buechiglasuster 0801e) for 90 min under the oil bath at 130˜150° C. Ethyl acetate (EA) was added to precipitate out the crude product in an ice water. The resulting precipitate was collected by filtration and purified by crystallization from n-hexane/EA to obtain Compound 20.

Compound 20 was obtained as red brown powder (yield 40%). R_{f}: 0.29 (ethyl acetate:n-hexane=2:1). mp 147-148° C. (EtOH). 1H-NMR (300 MHz, CDCl₃) δ ppm 2.79 (t, J=6.0 Hz, 4H, —NCH₂—), 3.02 (t, J=6.7 Hz, 4H, —CH₂CO—), 3.55 (s, 4H, —CH₂—), 3.72 (s, 6H, —OCH₃), 7.76 (t, J=8.4 Hz, 2H), 8.09 (d, J=9.0 Hz, 2H), 9.22 (d, J=9.6 Hz, 2H), 13.09 (s, 2H, Ar—NH—). 13C-NMR (75 MHz, CDCl₃) δ ppm 34.28, 45.37, 46.13, 51.73, 53.71, 64.45, 117.35, 122.61, 126.08, 134.68, 135.52, 140.89, 172.47 (NCO), 173.11 (CCO), 185.83 (CO). ESI-MS m/z: 525.3 [M+H]⁺.

### Example 21

### 1,5-Bis[2-(glycin methyl ester) propionamido]anthraquinone (Compound 21)

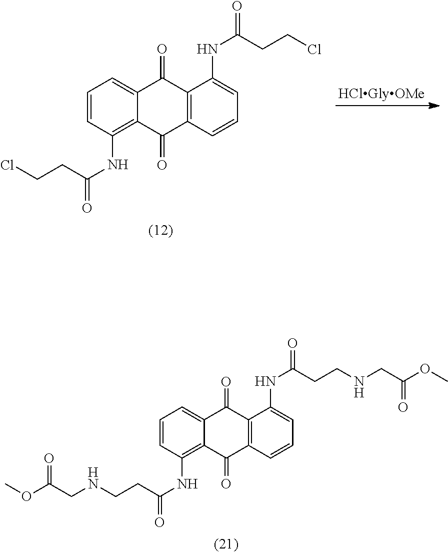

[Synthesis Process]:

DIPEA (1 ml, 6 mmole) was added in glycine methyl ester hydrochloride (0.37 g, 3 mmole) and the mixture was dissolved in N,N-dimethylforamide (20 mL). Next, Compound 12 (0.196 g, 0.5 mmol) was added. The mixture was then stirred in MiniClave—the compact autoclave (Buechiglasuster 0801e) for 90 min under the oil bath at 130˜150° C. Ethyl acetate (EA) was added to precipitate out the crude product in an ice water. The resulting precipitate was collected by filtration and purified by crystallization from n-hexane/EA to obtain Compound 21.

Compound 21 was obtained as red brown powder (yield 25%). R_{f}: 0.32 (ethyl acetate:n-hexane=2:1). mp 150-151° C. (EtOH). 1H-NMR (300 MHz, CDCl₃) δ ppm 2.74 (t, J=6.3 Hz, 4H, —CH₂N—), 3.08 (t, J=6.3 Hz, 4H, —COCH₂—), 3.51 (s, 4H, —CH₂—), 3.73 (s, 6H, —OCH₃), 7.77 (t, J=7.8 Hz, 2H), 8.03 (d, J=7.5 Hz, 2H), 9.13 (d, J=8.4 Hz, 2H), 12.35 (s, 2H, Ar—NH—). 13C-NMR (75 MHz, CDCl₃) δ ppm 38.91, 45.13, 50.76, 51.82, 116.98, 127.09, 129.21, 133.32, 134.43, 138.26, 171.57 (NCO), 172.73 (CCO), 186.85 (CO). HRMS (ESI) m/z calcd for C₂₆H₂₈N₄O₈ [M+H]⁺: 525.1907. Found: 525.1964.

### Example 22

### 2,6-Bis(chloroacetamido) anthraquinone (Compound 22)

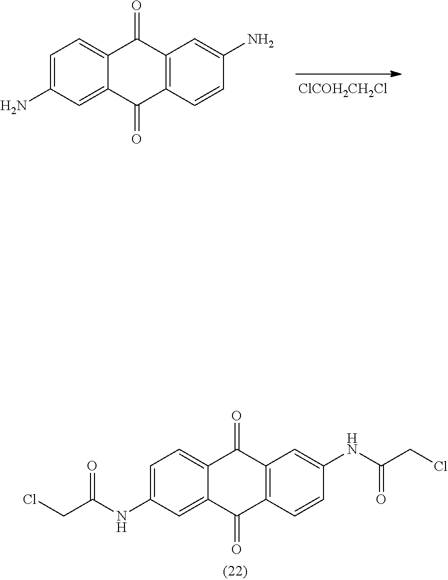

[Synthesis Process]:

2,6-diaminoanthraquinone (0.476 g, 2 mmol) was dissolved in N,N-dimethylforamide (20 ml) and pyridine (0.5 ml) under an ice water bath. Added chloroacetyl chloride (0.5 ml, 6 mmol), removed the ice water bath under nitrogen, protected from light, and stirred for 1 day at room temperature. The reaction mixture was poured into an ice water to precipitate out the crude product. The resulting precipitate was collected by filtration, washed with diethyl ether, and purified by crystallization from ethanol. Compound 22 was obtained

Yield: 75%; mp: 323-324° C. (EtOH) (lit32 mp: 323° C.). 1H-NMR (300 MHz, DMSO-d6) δ (ppm) 4.29 (s, 4H, —CH2-), 7.99 (d, J=6.9 Hz, 2H, H-4,8), 8.12 (d, J=8.4 Hz, 2H, H-3,7), 8.36 (s, 2H, H-1,5), 10.88 (s, 2H, —NH—).

### Example 23

### 2,6-Bis[2-(glycin methyl ester) acetamido]anthraquinone (Compound 23)

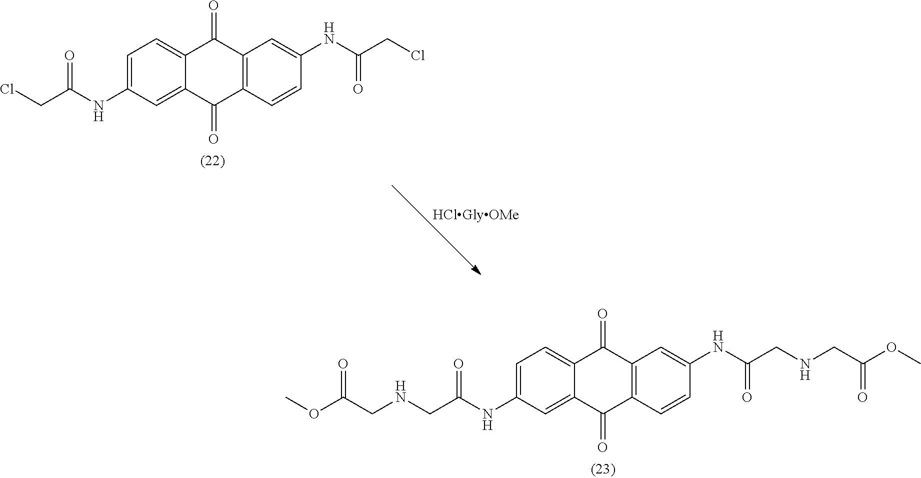

[Synthesis Process]:

DIPEA (1 ml, 6 mmole) was added in 2,6-bis(chloroacetamido) anthraquinone (22, 0.5 mmol) and glycine methyl ester hydrochloride) (0.37 g, 3 mmole). The mixture was dissolved in N,N-dimethylforamide (20 mL) and reacted for 48 hours at room temperature. Then, Ethyl acetate (EA) was added to precipitate out the crude product in an ice water to obtain Compound 23.

Compound 23 was obtained as yellow powder (yield 26%). R_{f}: 0.2 (ethyl acetate:n-hexane=1:1). mp 177-178° C. (EtOH). 1H-NMR (300 MHz, CDCl₃) δ ppm 3.49 (s, 4H, —CH₂—), 3.53 (s, 4H, —CH₂—), 3.77 (s, 6H, —OCH₃—), 8.15 (s, 2H, H-1,5), 8.27 (d, J=8.4 Hz, 2H, H-4,8), 8.36 (d, J=6.6 Hz, 2H, H-3,7), 9.86 (s, 2H, NH). 13C-NMR (75 MHz, CDCl₃) δ ppm 50.63, 51.98, 53.51, 117.71, 127.00, 128.82, 133.21, 134.20, 137.47, 171.58 (NCO), 172.52 (CCO), 186.35 (CO). HRMS (ESI) m/z calcd for C₂₄H₂₄N₄O₈ [M+H]⁺: 497.1594. Found: 497.1659.

### Example 24

### (L)-2,6-Bis[2-(alanine methyl ester) acetamido]anthraquinone (Compound 24)

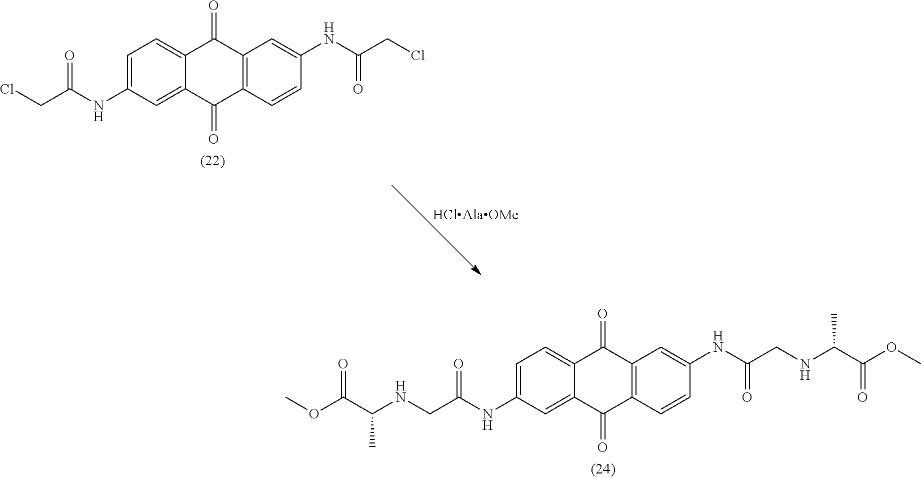

[Synthesis Process]:

DIPEA (1 ml, 6 mmole) was added in (L) alanine methyl ester hydrochloride (0.42 g, 3 mmole) and the mixture was dissolved in N,N-dimethylforamide (20 mL). Next, Compound 22 (0.196 g, 0.5 mmol) was added. The mixture was then stirred in MiniClave—the compact autoclave (Buechiglasuster 0801e) for 90 min under the oil bath at 130˜150° C. Ethyl acetate (EA) was added to precipitate out the crude product in an ice water. The resulting precipitate was collected by filtration and purified by crystallization from n-hexane/EA to obtain Compound 24.

Compound 24 was obtained as yellow powder (yield 36%). R_{f}: 0.28 (ethyl acetate:n-hexane=1:1). mp 131-132° C. (EtOH). 1H-NMR (300 MHz, CDCl₃) δ ppm 1.43 (d, J=6.9 Hz, 61-1, —CH₃), 3.33 (d, J=17.4 Hz, 2H, —CH₂—), 3.54 (d, J=17.7 Hz, 2H, —CH₂—), 3.41-3.48 (m, 2H, —CH—), 3.77 (s, 6H, —CH₃), 8.11 (s, 2H, H-1,5), 8.29 (d, J=8.7 Hz, 2H, H-4,8), 8.39 (d, J=9.0 Hz, 2H, H-3,7), 9.80 (s, 2H, —NH—). 13C-NMR (75 MHz, CDCl₃) δ ppm 19.11, 51.66, 52.34, 57.17, 116.57, 123.79, 129.27, 129.34, 134.86, 143.11, 170.10 (NCO), 175.09 (CCO), 181.79 (CO). HRMS (ESI) m/z calcd for C₂₆H₂₈N₄O₈ [M+H]⁺: 525.1907. Found: 525.1974.

### Example 25

### (D)-2,6-Bis[2-(alanine methyl ester) acetamido]anthraquinone (Compound 25)

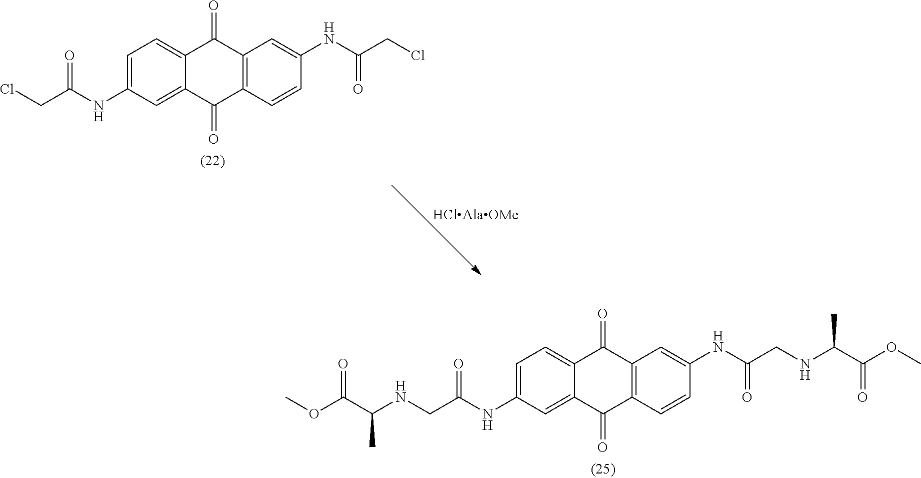

[Synthesis Process]:

DIPEA (1 ml, 6 mmole) was added in (D) alanine methyl ester hydrochloride (0.42 g, 3 mmole) and the mixture was dissolved in N,N-dimethylforamide (20 mL). Next, Compound 22 (0.196 g, 0.5 mmol) was added. The mixture was then stirred in MiniClave—the compact autoclave (Buechiglasuster 0801e) for 90 min under the oil bath at 130˜150° C. Ethyl acetate (EA) was added to precipitate out the crude product in an ice water. The resulting precipitate was collected by filtration and purified by crystallization from n-hexane/EA to obtain Compound 25.

Compound 25 was obtained as yellow powder (yield 33%). R_{f}: 0.28 (ethyl acetate:n-hexane=1:1). mp 131-132° C. (EtOH). 1H-NMR (300 MHz, CDCl₃) δ ppm 1.44 (d, J=6.9 Hz, 6H, —CH₃), 3.34 (d, J=17.4 Hz, 2H, —CH₂—), 3.55 (d, J=17.4 Hz, 2H, —CH₂—), 3.46 (dd, J=11.4, 6.3 Hz, 2H, —CH—), 3.78 (s, 6H, —CH₃), 8.12 (s, 2H, H-1,5), 8.29 (d, J=9.0 Hz, 2H, H-4,8), 8.39 (d, J=9.0 Hz, 2H, H-3,7), 9.81 (s, 2H, —NH—). 13C-NMR (75 MHz, CDCl₃) δ ppm 19.09, 51.65, 52.32, 57.15, 116.53, 123.75, 129.22, 129.28, 134.82, 143.09, 170.11 (NCO), 175.10 (CCO), 181.74 (CO). HRMS (ESI) m/z calcd for C₂₆H₂₈N₄O₈ [M+H]⁺: 525.1907. Found: 525.1964.

### Example 26

### (L)-2,6-Bis[2-(valine methyl ester) acetamido]anthraquinone (Compound 26)

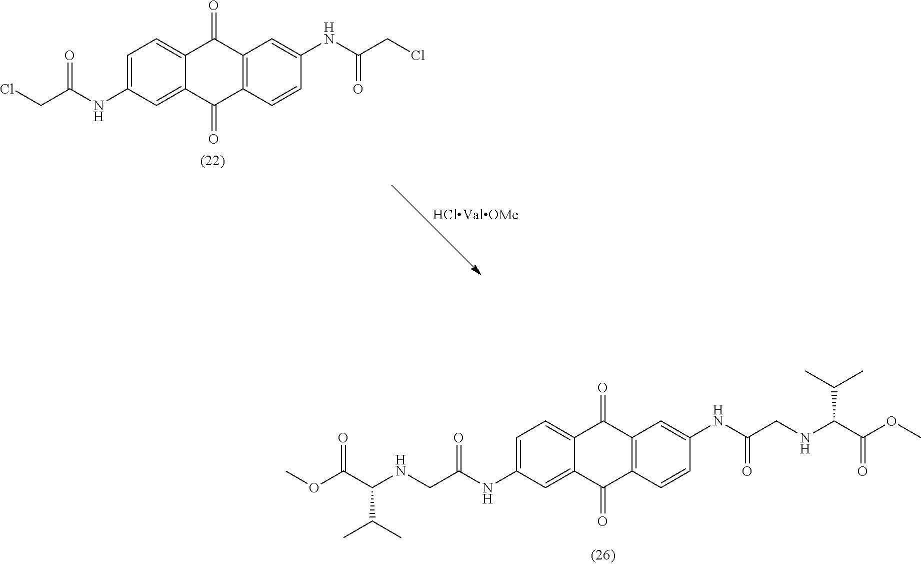

[Synthesis Process]:

DIPEA (1 ml, 6 mmole) was added in (L) valine methyl ester hydrochloride (0.50 g, 3 mmole) and the mixture was dissolved in N,N-dimethylforamide (20 mL). Next, Compound 22 (0.196 g, 0.5 mmol) was added. The mixture was then stirred in MiniClave—the compact autoclave (Buechiglasuster 0801e) for 90 min under the oil bath at 130˜150° C. Ethyl acetate (EA) was added to precipitate out the crude product in an ice water. The resulting precipitate was collected by filtration and purified by crystallization from n-hexane/EA to obtain Compound 26.

Compound 26 was obtained as yellow powder (yield 41%). R_{f}: 0.17 (ethyl acetate:n-hexane=1:1). mp 200-201° C. (EtOH). 1H-NMR (300 MHz, CDCl₃) δ ppm 1.03-1.10 (m, 12H, —CH₃), 2.07-2.13 (m, 2H, —CH—), 3.10 (d, J=5.4 Hz, 2H, —CH—), 3.19 (d, J=17.7 Hz, 2H —CH₂—), 3.60 (d, J=17.7 Hz, 2H, —CH₂—), 3.76 (s, 6H, —CH₃), 8.11 (s, 2H, H-1,4), 8.27 (d, J=8.4 Hz, 2H, H-4,8), 8.33 (d, J=8.7 Hz, 2H, H-3,7), 9.76 (s, 2H, —NH—). 13C-NMR (75 MHz, CDCl₃) δ ppm: 18.20, 19.69, 31.44, 52.02, 52.15, 67.83, 116.31, 123.62, 129.24, 134.83, 143.02, 170.06 (NCO), 174.55 (CCO), 181.61 (CO). HRMS (ESI) m/z calcd for C₃₀H₃₆N₄O₈ [M+H]⁺: 581.2533; Found: 581.2595.

### Example 27

### (D)-2,6-Bis[2-(valine methyl ester) acetamido anthraquinone (Compound 27)

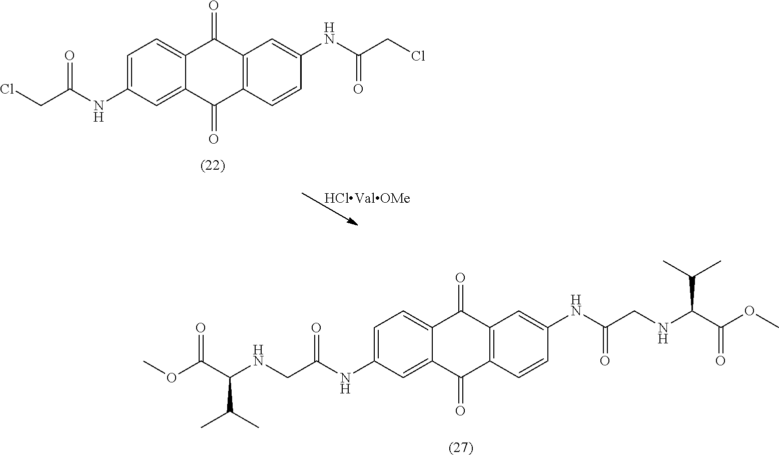

[Synthesis Process]:

DIPEA (1 ml, 6 mmole) was added in (D) valine methyl ester hydrochloride] (0.50 g, 3 mmole) and the mixture was dissolved in N,N-dimethylforamide (20 mL). Next, Compound 22 (0.196 g, 0.5 mmol) was added. The mixture was then stirred in MiniClave—the compact autoclave (Buechiglasuster 0801e) for 90 min under the oil bath at 130˜150° C. Ethyl acetate (EA) was added to precipitate out the crude product in an ice water. The resulting precipitate was collected by filtration and purified by crystallization from n-hexane/EA to obtain Compound 27.

Compound 27 was obtained as yellow powder (yield 43%). R_{f}: 0.17 (ethyl acetate:n-hexane=1:1). Mp 200-201° C. (EtOH). 1H-NMR (300 MHz, CDCl₃) δ ppm: 1.03-1.10 (m, 12H, —CH₃), 2.07-2.13 (m, 2H, —CH—), 3.10 (d, J=5.4 Hz, 2H, —CH—), 3.19 (d, J=17.7 Hz, 2H, —CH₂—), 3.60 (d, J=17.7 Hz, 2H, —CH₂—), 3.76 (s, 6H, —CH₃), 8.11 (s, 2H), 8.27 (d, J=8.4 Hz, 2H), 8.33 (d, J=8.7 Hz, 2H), 9.76 (s, 2H, —NH—). 13C-NMR (75 MHz, CDCl₃) δ ppm: 18.16, 19.65, 31.41, 51.99, 52.11, 67.79, 116.26, 123.56, 129.18, 134.77, 142.98, 170.04 (NCO), 174.52 (CCO), 181.54 (CO). HRMS (ESI) m/z calcd for C₃₀H₃₆N₄O₈ [M+H]⁺: 581.2533; Found: 581.2587.

### Example 28

### (L)-2,6-Bis[2-(leucine methyl ester) acetamido anthraquinone (Compound 28)

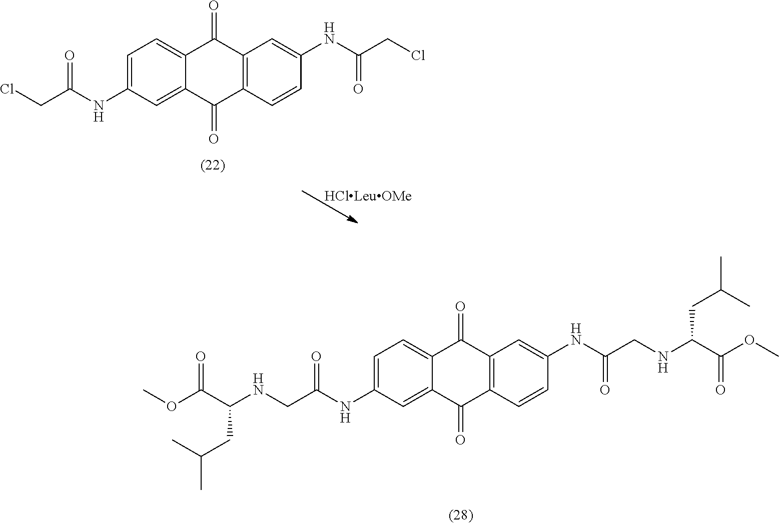

[Synthesis Process]:

DIPEA (1 ml, 6 mmole) was added in (L) leucine methyl ester hydrochloride] (0.55 g, 3 mmole) and the mixture was dissolved in N,N-dimethylforamide (20 mL). Next, Compound 22 (0.196 g, 0.5 mmol) was added. The mixture was then stirred in MiniClave—the compact autoclave (Buechiglasuster 0801e) for 90 min under the oil bath at 130˜150° C. Ethyl acetate (EA) was added to precipitate out the crude product in an ice water. The resulting precipitate was collected by filtration and purified by crystallization from n-hexane/EA to obtain Compound 28.

Compound 28 was obtained as yellowish brown powder (yield 39%). R_{f}: 0.26 (ethyl acetate:n-hexane=1:1). mp 179-180° C. (EtOH). 1H-NMR (300 MHz, CDCl₃) δ ppm: 0.96-1.02 (m, 12H, —CH₃), 1.55-1.92 (m, 6H, —CH₂—CH—), 3.25 (d, J=17.7 Hz, 2H, —CH₂—), 3.56 (d, J=17.7 Hz, 2H, —CH₂—), 3.35 (t, J=6.6 Hz, 2H, —CH—), 3.75 (s, 6H, —OCH₃), 8.10 (s, 2H), 8.27 (d, J=8.7 Hz, 2H), 8.34 (d, J=8.4 Hz, 2H), 9.77 (s, 2H, —NH—). 13C-NMR (75 MHz, CDCl₃) δ ppm: 21.85, 22.92, 25.11, 42.49, 51.80, 52.15, 60.39, 116.37, 123.60, 129.18, 129.21, 134.79, 142.99, 170.05 (NCO), 175.37 (CCO), 181.61 (CO). HRMS (ESI) m/z calcd for C₃₂H₄₀N₄O₈ [M+H]⁺: 609.2846; Found: 609.2911.

### Example 29

### 2,6-Bis[2-(sarcosine methyl ester) acetamido]anthraquinone (Compound 29)

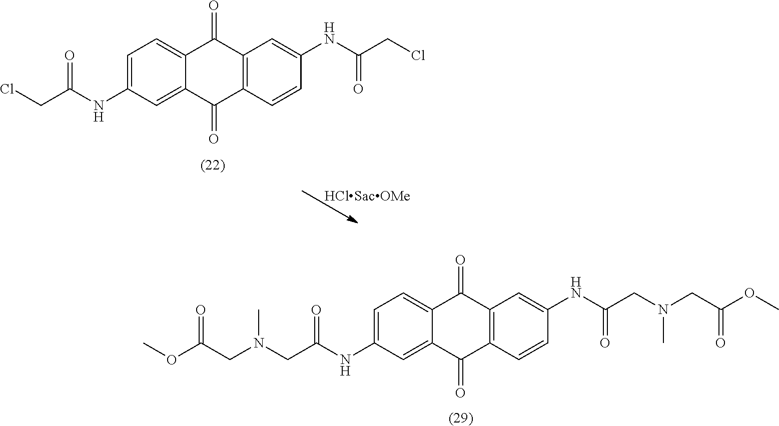

[Synthesis Process]:

DIPEA (1 ml, 6 mmole) was added in sarcosine methyl ester hydrochloride) (0.42 g, 3 mmole) and the mixture was dissolved in N,N-dimethylforamide (20 mL). Next, Compound 22 (0.196 g, 0.5 mmol) was added. The mixture was then stirred in MiniClave—the compact autoclave (Buechiglasuster 0801e) for 90 min under the oil bath at 130˜150° C. Ethyl acetate (EA) was added to precipitate out the crude product in an ice water. The resulting precipitate was collected by filtration and purified by crystallization from n-hexane/EA to obtain Compound 29.

Compound 29 was obtained as yellow powder (yield 58%). R_{f}: 0.23 (ethyl acetate:n-hexane=1:1). mp 145-146° C. (EtOH). 1H-NMR (300 MHz, CDCl₃) δ ppm: 2.55 (s, 6H, —N—CH₃), 3.36 (s, 4H, —CH₂—), 3.46 (s, 4H, —CH₂—), 3.79 (s, 6H, —OCH₃), 8.20 (s, 2H, H-1,4), 8.28 (d, J=8.7 Hz, 2H), 8.37 (d, J=8.4 Hz, 2H), 10.03 (s, 2H, —NH—). 13C-NMR (75 MHz, CDCl₃) δ ppm: 51.44, 52.71, 65.75, 116.58, 123.73, 127.43, 129.18, 134.70, 137.10, 142.97, 169.89 (NCO), 172.82 (CCO), 181.64 (CO). ESI-MS m/z: 525.3 [M+H]⁺.

### Example 30

### (S)-2,6-Bis[2-(phenylglycin methyl ester) acetamido]anthraquinone (Compound 30)

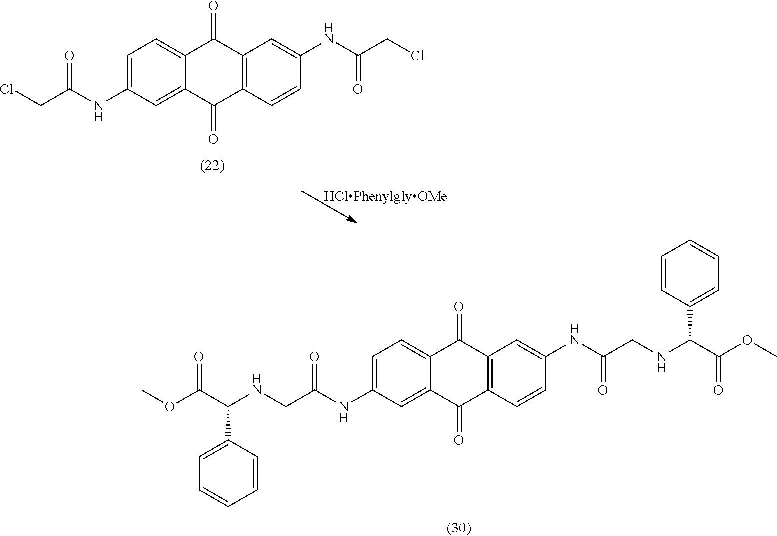

[Synthesis Process]:

DIPEA (1 ml, 6 mmole) was added in (S) phenylglycin methyl ester hydrochloride (0.61 g, 3 mmole) and the mixture was dissolved in N,N-dimethylforamide (20 mL). Next, Compound 22 (0.196 g, 0.5 mmol) was added. The mixture was then stirred in MiniClave—the compact autoclave (Buechiglasuster 0801e) for 90 min under the oil bath at 130˜150° C. Ethyl acetate (EA) was added to precipitate out the crude product in an ice water. The resulting precipitate was collected by filtration and purified by crystallization from n-hexane/EA to obtain Compound 30.

Compound 30 was obtained as yellowish brown powder (yield 44%). R_{f}: 0.23 (ethyl acetate:n-hexane=1:1). mp 210-211° C. (EtOH). 1H-NMR (300 MHz, CDCl₃) δ ppm: 3.42 (d, J=17.4 Hz, 2H, —CH₂—), 3.51 (d, J=17.7 Hz, 2H, —CH₂—), 3.75 (s, 6H, —CH₃), 4.43 (s, 2H, —CH—), 7.39 (s, 10H, —C₆H₅), 8.06 (s, 2H), 8.27 (d, J=4.2 Hz, 4H), 9.68 (s, 2H, —NH—). 13C-NMR (75 MHz, CDCl₃) δ ppm: 51.46, 52.73, 65.79, 116.60, 123.76, 127.43, 128.89, 129.18, 129.25, 134.76, 137.08, 142.99, 169.83 (NCO), 172.79 (CCO), 181.69 (CO). HRMS (ESI) m/z calcd for C₃₆H₃₂N₄O₈ [M+H]⁺: 649.2220; Found: 649.2307.

### Example 31

### (R)-2,6-Bis[2-(phenylglycin methyl ester) acetamido]anthraquinone (Compound 31)

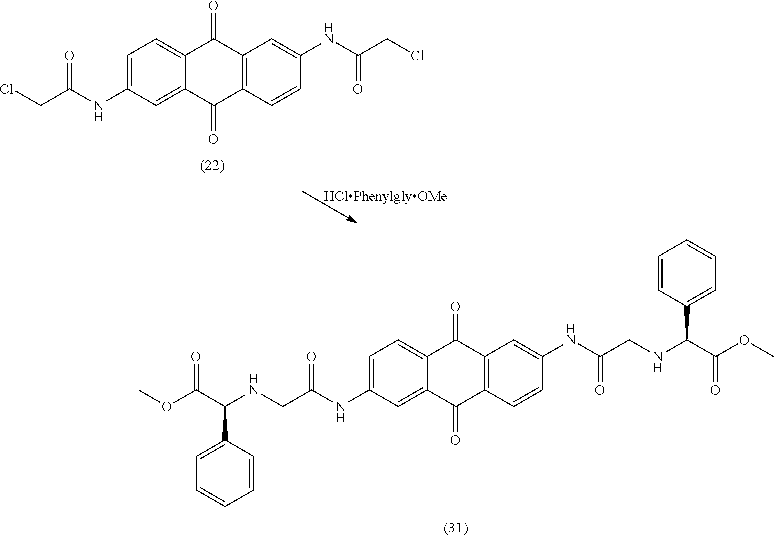

[Synthesis Process]:

DIPEA (1 ml, 6 mmole) was added in (R) phenylglycin methyl ester hydrochloride] (0.61 g, 3 mmole) and the mixture was dissolved in N,N-dimethylforamide (20 mL). Next, Compound 22 (0.196 g, 0.5 mmol) was added. The mixture was then stirred in MiniClave—the compact autoclave (Buechiglasuster 0801e) for 90 min under the oil bath at 130˜150° C. Ethyl acetate (EA) was added to precipitate out the crude product in an ice water. The resulting precipitate was collected by filtration and purified by crystallization from n-hexane/EA to obtain Compound 31.

Compound 31 was obtained as yellow powder (yield 35%). R_{f}: 0.22 (ethyl acetate:n-hexane=1:1). mp 202-203° C. (EtOH). 1H-NMR (300 MHz, CDCl₃) δ ppm: 3.39 (d, J=17.1 Hz, 2H, —CH₂—), 3.48 (d, J=18.0 Hz, 2H, —CH₂—), 3.73 (s, 6H, —CH₃), 4.41 (s, 2H, —CH—), 7.37 (s, 10H, —C₆H₅), 8.04 (s, 2H), 8.24 (d, J=3.6 Hz, 4H), 9.66 (s, 2H, —NH—). 13C-NMR (75 MHz, CDCl₃) δ ppm: 51.32, 51.37, 53.46, 116.68, 123.08, 128.55, 128.77, 129.02, 129.12, 134.53, 137.39, 142.66, 169.58 (NCO), 174.34 (CCO), 181.54 (CO). HRMS (ESI) m/z calcd for C₃₆H₃₂N₄O₈ [M+H]⁺: 649.2220; Found: 649.2263.

### Example 32

### 2,6-Bis[2-(phenylalanine methyl ester) acetamido]anthraquinone (Compound 32)

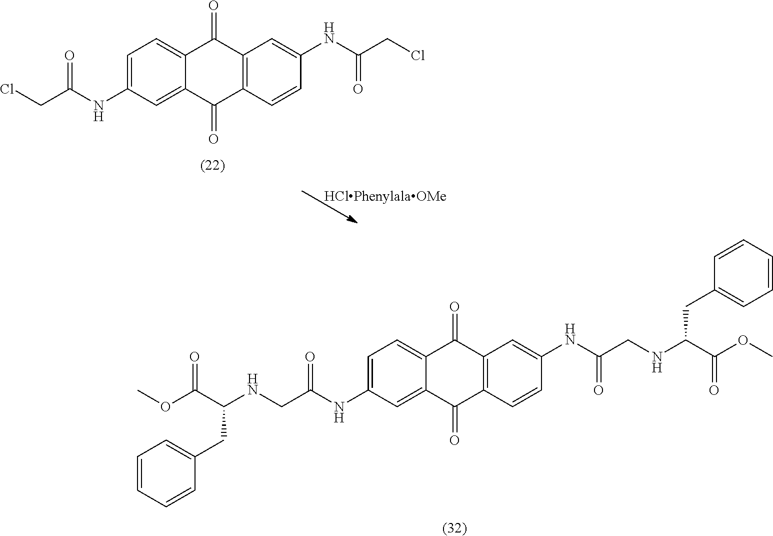

[Synthesis Process]:

DIPEA (1 ml, 6 mmole) was added in phenylalanine methyl ester hydrochloride (0.65 g, 3 mmole) and the mixture was dissolved in N,N-dimethylforamide (20 mL). Next, Compound 22 (0.196 g, 0.5 mmol) was added. The mixture was then stirred in MiniClave—the compact autoclave (Buechiglasuster 0801e) for 90 min under the oil bath at 130˜150° C. Ethyl acetate (EA) was added to precipitate out the crude product in an ice water. The resulting precipitate was collected by filtration and purified by crystallization from n-hexane/EA to obtain Compound 32.

Compound 32 was obtained as yellowish brown powder (yield 25%). R_{f}: 0.32 (ethyl acetate:n-hexane=1:1). mp 197-198° C. (EtOH). 1H-NMR (300 MHz, CDCl₃) δ ppm: 2.75 (t, J=12.9 Hz, 2H, —CH—), 3.16 (d, J=18.0 Hz, 2H, —CH₂—), 3.55 (d, J=18.0 Hz, 2H, —CH₂—), 3.79 (s, 6H, —CH₃), 7.27-7.42 (m, 10H, —C₆H₅), 7.72 (s, 2H), 7.93 (d, J=8.7 Hz, 2H), 8.19 (d, J=8.7 Hz, 2H), 9.08 (s, 2H, —NH—). 13C-NMR (75 MHz, CDCl₃) δ ppm: 39.51, 51.51, 52.37, 63.46, 116.67, 123.68, 127.55, 129.02, 134.52, 137.19, 143.66, 169.89 (NCO), 174.40 (CCO), 181.54 (CO). HRMS (ESI) m/z calcd for C₃₈H₃₆N₄O₈ [M+H]⁺: 677.2533; Found: 677.2593.

### Example 33

### [2,7-Bis(chloroacetamido) anthraquinone] (Compound 33)

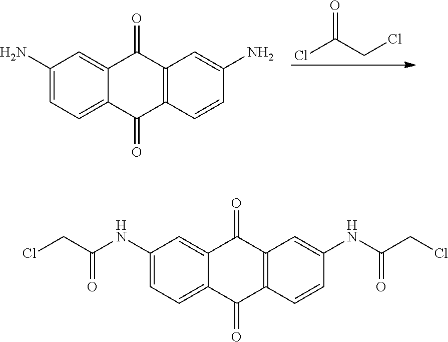

[Synthesis Process]:

2,7-diaminoanthraquinone (0.476 g, 2 mmol) was dissolved in N,N-dimethylforamide (20 ml) and pyridine (0.5 ml) under an ice water bath. Added chloroacetyl chloride (0.5 ml, 6 mmol), removed the ice water bath under nitrogen, protected from light, and stirred for 1 day at room temperature. The reaction mixture was poured into an ice water to precipitate out the crude product. The resulting precipitate was collected by filtration, washed with diethyl ether, and purified by crystallization from ethanol. Compound 33 was obtained, Yield: 60%. mp: 266-267° C. (EtOH). 1H-NMR (300 MHz, DMSO-d₆) δ ppm 4.33 (s, 4H, —CH₂—), 8.02 (dd, J=8.7, 2.4 Hz, 2H), 8.13 (d, J=8.7 Hz, 2H), 8.40 (d, J=2.1 Hz, 2H), 10.90 (s, 2H, —NH—).

### Example 34

### 2,7-Bis[2-(glycin methyl ester) acetamido]anthraquinone (Compound 34)

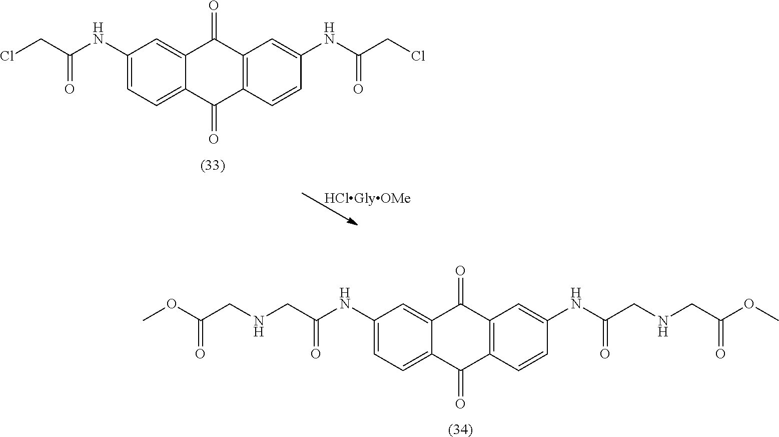

[Synthesis Process]:

DIPEA (1 ml, 6 mmole) was added in 2,7-bis(chloroacetamido) anthraquinone (33, 0.5 mmol) and glycine methyl ester hydrochloride) (0.37 g, 3 mmole). The mixture was dissolved in N,N-dimethylforamide (20 mL) and reacted for 48 hours at room temperature. Then, Ethyl acetate (EA) was added to precipitate out the crude product in an ice water to obtain Compound 34.

Compound 34 was obtained as yellow powder (yield 26%). R_{f}: 0.23 (ethyl acetate:n-hexane=2:1). mp 147-148° C. (EtOH). 1H-NMR (300 MHz, CDCl₃) δ ppm: 3.49 (s, 4H, —CH₂—), 3.54 (s, 4H, —CH₂—), 3.77 (s, 6H, —OCH₃), 8.19 (s, 2H, H-1,8), 8.28 (d, J=8.1 Hz, 2H), 8.32 (d, J=9.0 Hz, 2H), 9.84 (s, 2H, —NH—). 13C-NMR (75 MHz, CDCl₃) δ ppm: 50.63, 51.98, 53.51, 117.71, 127.00, 128.82, 133.21, 134.20, 137.47, 171.58 (NCO), 172.52 (CCO), 186.35 (CO). HRMS (ESI) m/z calcd for C₂₄H₂₄N₄O₈ [M+H]⁺: 497.1594; Found: 497.1654.

### Example 35

### (L)-2,7-Bis[2-(alanine methyl ester) acetamido]anthraquinone (Compound 35)

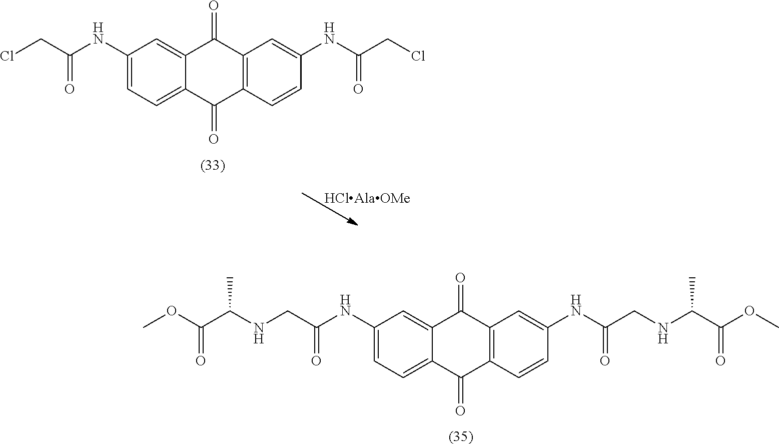

[Synthesis Process]:

DIPEA (1 ml, 6 mmole) was added in (L) alanine methyl ester hydrochloride] (0.42 g, 3 mmole) and the mixture was dissolved in N,N-dimethylforamide (20 mL). Next, Compound 33 (0.196 g, 0.5 mmol) was added. The mixture was then stirred in MiniClave—the compact autoclave (Buechiglasuster 0801e) for 90 min under the oil bath at 130˜150° C. Ethyl acetate (EA) was added to precipitate out the crude product in an ice water. The resulting precipitate was collected by filtration and purified by crystallization from n-hexane/EA to obtain Compound 35.

Compound 35 was obtained as yellow powder (yield 30%). R_{f}: 0.28 (ethyl acetate:n-hexane=2:1). Mp 147-148° C. (EtOH). 1H-NMR (300 MHz, CDCl₃) δ ppm: 1.43 (d, J=6.9 Hz, 6H, —CH₃), 3.33 (d, J=17.4 Hz, 2H, —CH₂—), 3.54 (d, J=17.1 Hz, 2H, —CH₂—), 3.45 (dd, J=13.8, 6.9 Hz, 2H, —CH—), 3.76 (s, 6H, —CH₃), 8.15 (s, 2H, H-1,8), 8.28 (d, J=8.4 Hz, 2H), 8.33 (d, J=8.4 Hz, 2H), 9.78 (s, 2H, —NH—). 13C-NMR (75 MHz, CDCl₃) δ ppm: 19.08, 51.63, 52.31, 57.13, 116.52, 124.14, 129.19, 129.35, 134.60, 142.73, 170.12 (NCO), 175.11 (CCO), 182.68 (CO). HRMS (ESI) m/z calcd for C₂₆H₂₈N₄O₈ [M+H]⁺: 525.1907; Found: 525.1975.

### Example 36

### (D)-2,7-Bis[2-(alanine methyl ester) acetamido]anthraquinone (Compound 36)

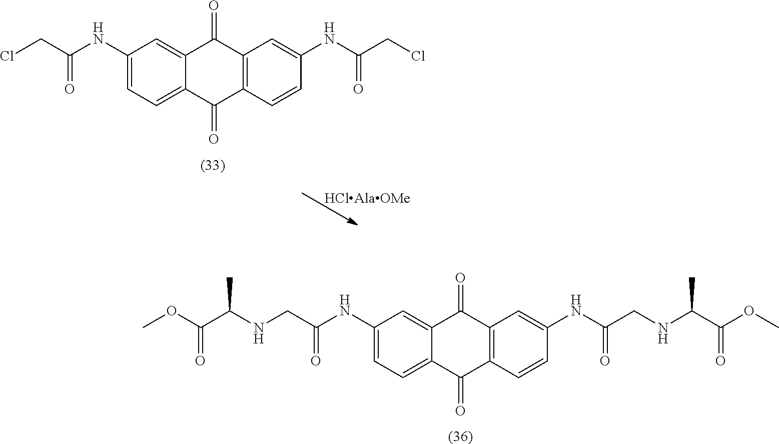

[Synthesis Process]:

DIPEA (1 ml, 6 mmole) was added in (D) alanine methyl ester hydrochloride] (0.42 g, 3 mmole) and the mixture was dissolved in N,N-dimethylforamide (20 mL). Next, Compound 33 (0.196 g, 0.5 mmol) was added. The mixture was then stirred in MiniClave—the compact autoclave (Buechiglasuster 0801e) for 90 min under the oil bath at 130˜150° C. Ethyl acetate (EA) was added to precipitate out the crude product in an ice water. The resulting precipitate was collected by filtration and purified by crystallization from n-hexane/EA to obtain Compound 36.

Compound 36 was obtained as yellow powder (yield 25%). R_{f}: 0.28 (ethyl acetate:n-hexane=1:2). mp 145-146° C. (EtOH). 1H-NMR (300 MHz, CDCl₃) δ ppm: 1.44 (d, J=6.6 Hz, 6H, —CH₃), 3.34 (d, J=17.7 Hz, 2H, —CH₂—), 3.56 (d, J=17.1 Hz, 2H, —CH₂—), 3.46 (dd, J=13.8, 6.9 Hz, 2H, —CH—), 3.78 (s, 6H, —CH₃), 8.16 (s, 2H, H-1,8), 8.30 (d, J=8.4 Hz, 2H, H-4,5), 8.35 (d, J=8.7 Hz, 2H, H-3,6), 9.79 (s, 2H, —NH—). 13C-NMR (75 MHz, CDCl₃) δ ppm: 19.08, 51.63, 52.31, 57.12, 116.50, 124.11, 129.17, 129.33, 134.59, 142.73, 170.13 (NCO), 175.11 (CCO), 182.65 (CO). HRMS (ESI) m/z calcd for C₂₆H₂₈N₄O₈ [M+H]⁺: 525.1907; Found: 525.1963.

### Example 37

### (L)-2,7-Bis[2-(valine methyl ester) acetamido]anthraquinone (Compound 37)

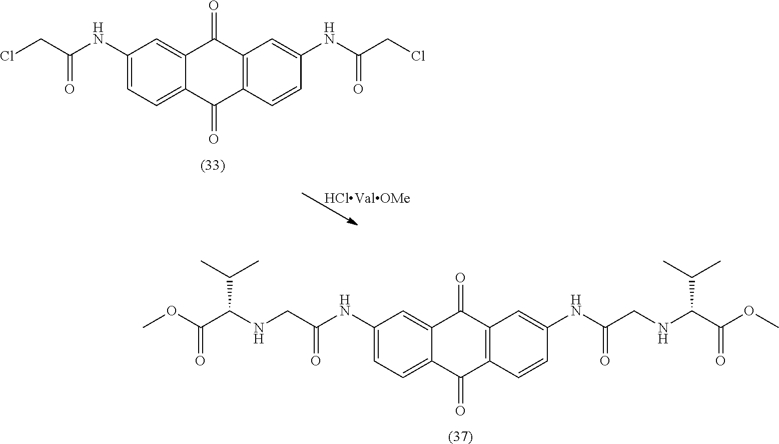

[Synthesis Process]:

DIPEA (1 ml, 6 mmole) was added in (L) valine methyl ester hydrochloride] (0.50 g, 3 mmole) and the mixture was dissolved in N,N-dimethylforamide (20 mL). Next, Compound 33 (0.196 g, 0.5 mmol) was added. The mixture was then stirred in MiniClave—the compact autoclave (Buechiglasuster 0801e) for 90 min under the oil bath at 130˜150° C. Ethyl acetate (EA) was added to precipitate out the crude product in an ice water. The resulting precipitate was collected by filtration and purified by crystallization from n-hexane/EA to obtain Compound 37.

Compound 37 was obtained as yellow powder (yield 45%). R_{f}: 0.30 (ethyl acetate:n-hexane=2:1). mp 191-192° C. (EtOH). 1H-NMR (300 MHz, CDCl₃) δ ppm: 1.04-1.11 (m, 12H, —CH₃), 2.13 (d, J=5.4 Hz, 2H, —CH—), 3.12 (s, 2H, —CH—), 3.22 (d, J=17.7 Hz, 2H —CH₂—), 3.62 (d, J=17.4 Hz, 2H, —CH₂—), 3.77 (s, 6H, —CH₃), 8.15 (s, 2H, H-1,8), 8.28 (s, 4H, H-4,3,5,6), 9.78 (s, 2H, —NH—). 13C-NMR (75 MHz, CDCl₃) δ ppm: 18.16, 19.68, 31.42, 52.02, 52.12, 67.81, 116.28, 123.95, 129.17, 129.32, 134.64, 142.67, 170.53 (NCO), 174.55 (CCO), 182.49 (CO). HRMS (ESI) m/z calcd for C₃₀H₃₆N₄O₈ [M+H]⁺: 581.2533; Found: 581.2608.

### Example 38

### (D)-2,7-Bis[2-(valine methyl ester) acetamido]anthraquinone (Compound 38)

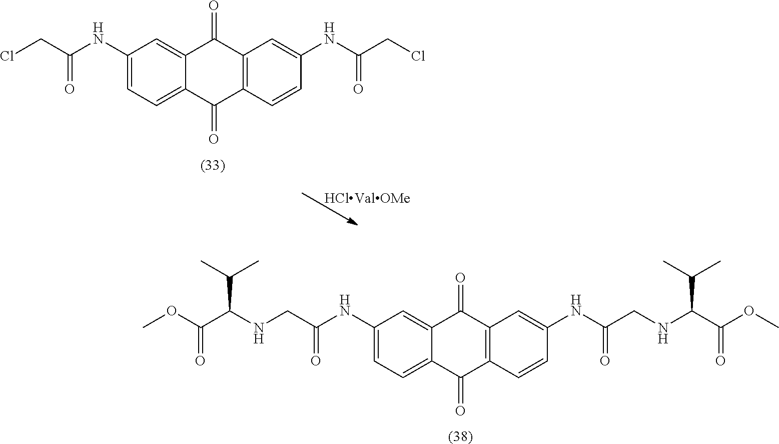

[Synthesis Process]:

DIPEA (1 ml, 6 mmole) was added in (D) valine methyl ester hydrochloride (0.50 g, 3 mmole) and the mixture was dissolved in N,N-dimethylforamide (20 mL). Next, Compound 33 (0.196 g, 0.5 mmol) was added. The mixture was then stirred in MiniClave—the compact autoclave (Buechiglasuster 0801e) for 90 min under the oil bath at 130˜150° C. Ethyl acetate (EA) was added to precipitate out the crude product in an ice water. The resulting precipitate was collected by filtration and purified by crystallization from n-hexane/EA to obtain Compound 38.

Compound 38 was obtained as yellow powder (yield 40%). R_{f}: 0.30 (ethyl acetate:n-hexane=2:1). mp 192-193° C. (EtOH). 1H-NMR (300 MHz, CDCl₃) δ ppm: 1.04-1.11 (m, 12H, —CH₃), 2.12 (s, 2H, —CH—), 3.13 (s, 2H, —CH—), 3.23 (d, J=19.2 Hz, 2H —CH₂—), 3.63 (d, 18.6 Hz, 2H, —CH₂—), 3.77 (s, 6H, —CH₃), 8.14 (s, 2H, H-1,8), 8.27 (s, 4H, H-4,3,5,6), 9.79 (s, 2H, —NH—). 13C-NMR (75 MHz, CDCl₃) δ ppm: 17.55, 18.89, 30.82, 51.30, 51.60, 67.36, 115.89, 123.60, 128.71, 129.05, 134.38, 142.32, 169.36 (NCO), 173.94 (CCO), 182.16 (CO). HRMS (ESI) m/z calcd for C₃₀H₃₆N₄O₈ [M+H]⁺: 581.2533; Found: 581.2599.

### Example 39

### (S)-2,7-Bis[2-(phenylglycin methyl ester) acetamido]anthraquinone (Compound 39)

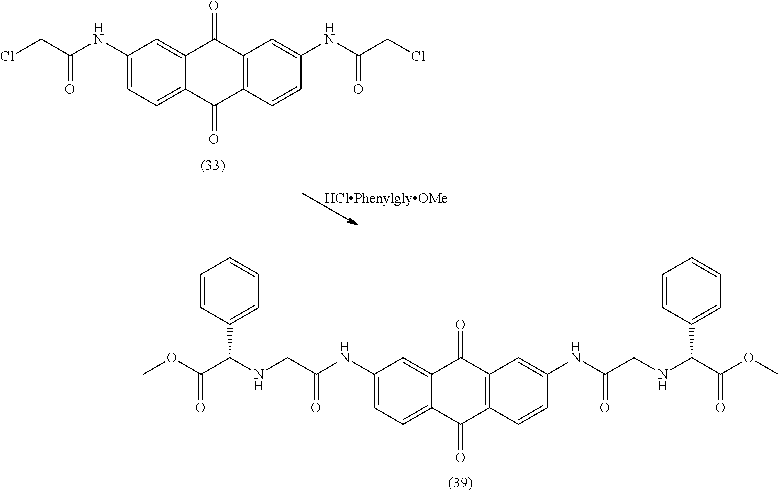

[Synthesis Process]:

DIPEA (1 ml, 6 mmole) was added in (S) phenylglycin methyl ester hydrochloride (0.61 g, 3 mmole) and the mixture was dissolved in N,N-dimethylforamide (20 mL). Next, Compound 33 (0.196 g, 0.5 mmol) was added. The mixture was then stirred in MiniClave—the compact autoclave (Buechiglasuster 0801e) for 90 min under the oil bath at 130˜150° C. Ethyl acetate (EA) was added to precipitate out the crude product in an ice water. The resulting precipitate was collected by filtration and purified by crystallization from n-hexane/EA to obtain Compound 39.

Compound 39 was obtained as yellowish brown powder (yield 32%). R_{f}: 0.32 (ethyl acetate:n-hexane=2:1). mp 160-161° C. (EtOH). 1H-NMR (300 MHz, CDCl₃) δ ppm: 3.41 (d, J=17.1 Hz, 2H, —CH₂—), 3.50 (d, J=17.7 Hz, 2H, —CH₂—), 3.75 (s, 6H, —CH₃), 4.43 (s, 2H, —CH—), 7.39 (s, 10H, —C₆H₅), 8.08 (s, 2H, H-1,8), 8.25 (s, 4H, H-4,3,5,6), 9.66 (s, 2H, —NH—). 13C-NMR (75 MHz, CDCl₃) δ ppm: 51.46, 52.73, 65.79, 116.60, 123.76, 127.43, 129.18, 134.76, 137.08, 142.99, 169.83 (NCO), 172.79 (CCO), 181.69 (CO). HRMS (ESI) m/z calcd for C₃₆H₃₂N₄O₈ [M+H]⁺: 649.2220; Found: 649.2272.

### Example 40

### (R)-2,7-Bis[2-(phenylglycin methyl ester) acetamido]anthraquinone (Compound 40)

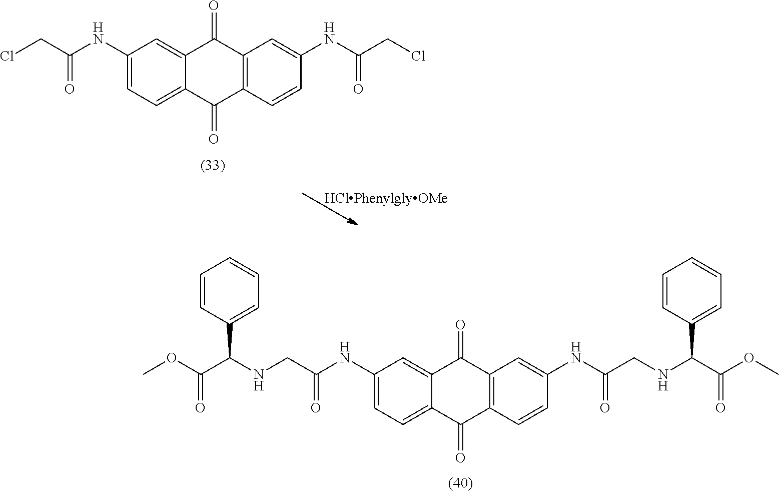

[Synthesis Process]:

DIPEA (1 ml, 6 mmole) was added in (R) phenylglycin methyl ester hydrochloride] (0.61 g, 3 mmole) and the mixture was dissolved in N,N-dimethylforamide (20 mL). Next, Compound 33 (0.196 g, 0.5 mmol) was added. The mixture was then stirred in MiniClave—the compact autoclave (Buechiglasuster 0801e) for 90 min under the oil bath at 130˜150° C. Ethyl acetate (EA) was added to precipitate out the crude product in an ice water. The resulting precipitate was collected by filtration and purified by crystallization from n-hexane/EA to obtain Compound 40.

Compound 40 was obtained as yellow powder (yield 35%). R_{f}: 0.32 (ethyl acetate:n-hexane=2:1). mp 165-166° C. (EtOH). 1H-NMR (300 MHz, CDCl₃) δ ppm: 3.43 (d, J=6.0 Hz, 2H —CH₂—), 3.51 (d, J=16.5 Hz, 2H, —CH₂—), 3.75 (s, 6H, —CH₃), 4.44 (s, 2H, —CH—), 7.40 (s, 10H, —C₆H₅), 8.10 (s, 2H, H-1,8), 8.25 (s, 4H, H-4,3,5,6), 9.68 (s, 2H, —NH—). 13C-NMR (75 MHz, CDCl₃) δ ppm: 51.43, 52.72, 65.75, 116.57, 124.10, 127.42, 129.07, 134.53, 137.11, 142.65, 169.89 (NCO), 172.83 (CCO), 182.54 (CO). HRMS (ESI) m/z calcd for C₃₆H₃₂N₄O₈ [M+H]⁺: 649.2220; Found: 649.2285.

### Example 41

### 2,7-Bis[2-(phenylalanine methyl ester) acetamido]anthraquinone (Compound 41)

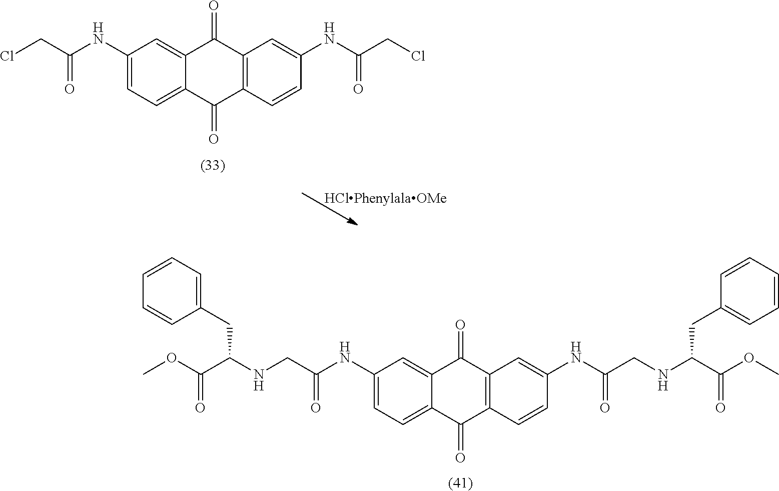

[Synthesis Process]:

DIPEA (1 ml, 6 mmole) was added in phenylalanine methyl ester hydrochloride (0.65 g, 3 mmole) and the mixture was dissolved in N,N-dimethylforamide (20 mL). Next, Compound 33 (0.196 g, 0.5 mmol) was added. The mixture was then stirred in MiniClave—the compact autoclave (Buechiglasuster 0801e) for 90 min under the oil bath at 130˜150° C. Ethyl acetate (EA) was added to precipitate out the crude product in an ice water. The resulting precipitate was collected by filtration and purified by crystallization from n-hexane/EA to obtain Compound 41.

Compound 41 was obtained as yellow powder (yield 42%). R_{f}: 0.35 (ethyl acetate: n-hexane=2:1). mp 191-192° C. (EtOH). 1H-NMR (300 MHz, CDCl₃) δ ppm: 2.76 (t, J=10.2 Hz, 2H —CH—), 3.18 (d, J=18.0 Hz, 2H —CH₂—), 3.57 (d, J=17.7 Hz, 2H, —CH₂—), 3.81 (s, 6H, —CH₃), 7.28-7.43 (m, 10H, —C₆H₅), 7.78 (s, 2H, H-1,8), 7.88 (d, J=6.3 Hz, 2H, H-4,5), 8.18 (d, J=8.4 Hz, 2H, H-3,6), 9.09 (s, 2H, —NH—). 13C-NMR (75 MHz, CDCl₃) δ ppm: 39.56, 51.51, 52.38, 63.49, 116.65, 123.94, 127.57, 129.06, 134.46, 137.24, 142.40, 169.86 (NCO), 174.41 (CCO), 182.20 (CO). HRMS (ESI) m/z calcd for C₃₈H₃₆N₄O₈ [M+H]⁺: 677.2533; Found: 677.2600.

TABLE 1
Compounds 1~10

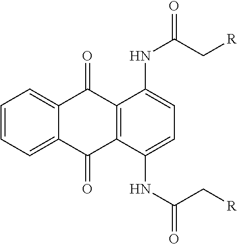

| | | | | Yield |
|---|---|---|---|---|
| No. | R | M.w. | M.p. | % |
| 1 | Cl | 391.20 | 284-285 | 75 |
| 2 | CH₃Cl | 406.20 | 225-226 | 44 |
| 3 | NHCH₂CO₂CH₃ | 496.47 | 164-165 | 30 |
| 4 | (L)-NHCH(CH₃)CO₂CH₃ | 524.52 | 149-150 | 26 |
| 5 | (D)-NHCH(CH₃)CO₂CH₃ | 524.52 | 149-150 | 33 |
| 6 | (L)-NHCH(CH(CH₃)CH₃)CO₂CH₃ | 580.63 | 180-181 | 30 |
| 7 | (D)-NHCH(CH(CH₃)CH₃)CO₂CH₃ | 580.63 | 180-181 | 43 |
| 8 | (L)-NHCH(CH₂CH(CH₃)CH₃)CO₂CH₃ | 608.68 | 200-201 | 39 |
| 9 | N(CH₃)CH₂CO₂CH₃ | 524.52 | 150-151 | 45 |
| 10 | CH₃NHCH₂CO₂CH₃ | 524.52 | 155-156 | 23 |

TABLE 2
Compounds 11~21

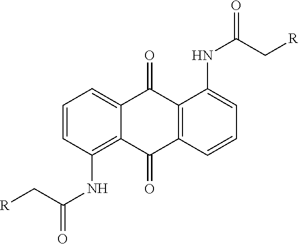

| | | | | Yield |
|---|---|---|---|---|
| No. | R | M.w. | M.p. | % |
| 11 | Cl | 391.20 | 349-350 | 80 |
| 12 | CH₃Cl | 406.20 | 275-276 | 45 |
| 13 | (L)-NHCH(CH₃)CO₂CH₃ | 524.52 | 141-142 | 40 |
| 14 | (D)-NHCH(CH₃)CO₂CH₃ | 524.52 | 143-144 | 45 |
| 15 | (L)-NHCH(CH(CH₃)CH₃)CO₂CH₃ | 580.63 | 125-126 | 35 |
| 16 | (D)-NHCH(CH(CH₃)CH₃)CO₂CH₃ | 580.63 | 126-127 | 41 |
| 17 | (L)-NHCH(CH₂CH(CH₃)CH₃)CO₂CH₃ | 608.68 | 149-150 | 35 |
| 18 | (L)-NHCH(CO₂CH₃)CH₂CH₂CO₂CH₃ | 668.65 | 151-152 | 38 |
| 19 | N(CH₃)CH₂CO₂CH₃ | 524.52 | 186-187 | 55 |
| 20 | NHCH₂CH₂CO₂CH₃ | 524.52 | 147-148 | 40 |
| 21 | CH₂NHCH₂CO₂CH3 | 524.52 | 150-151 | 25 |

TABLE 3
Compounds 22~32

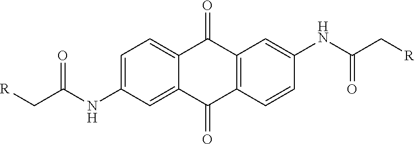

| | | | | Yield |
|---|---|---|---|---|
| No. | R | M. w. | M. p. | % |
| 22 | Cl | 391.20 | 323-324 | 75 |
| 23 | NHCH₂CO₂CH₃ | 496.47 | 177-178 | 26 |
| 24 | (L)-NHCH(CH₃)CO₂CH₃ | 524.52 | 131-132 | 36 |
| 25 | (D)-NHCH(CH₃)CO₂CH₃ | 524.52 | 131-132 | 33 |
| 26 | (L)-NHCH(CH(CH₃)CH₃)CO₂CH₃ | 580.63 | 200-201 | 41 |
| 27 | (D)-NHCH(CH(CH₃)CH₃)CO₂CH₃ | 580.63 | 200-201 | 43 |
| 28 | (L)-NHCH(CH₂CH(CH₃)CH₃)CO₂CH₃ | 608.68 | 179-180 | 39 |
| 29 | N(CH₃)CH₂CO₂CH₃ | 524.52 | 145-146 | 58 |
| 30 | (L)-NHCH(C₆H₅)CO₂CH₃ | 648.66 | 210-211 | 44 |
| 31 | (D)-NHCH(C₆H₅)CO₂CH₃ | 648.66 | 202-203 | 35 |
| 32 | (L)-NHCH(CH₂C₆H₅)CO₂CH₃ | 676.25 | 197-198 | 25 |

TABLE 4
Compounds 33~41

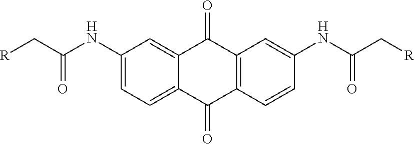

| | | | | Yield |
|---|---|---|---|---|
| No. | R | M. w. | M. p. | % |
| 33 | Cl | 391.20 | 266-267 | 60 |
| 34 | NHCH₂CO₂CH₃ | 496.47 | 147-148 | 26 |
| 35 | (L)-NHCH(CH₃)CO₂CH₃ | 524.52 | 147-148 | 30 |
| 36 | (D)-NHCH(CH₃)CO₂CH₃ | 524.52 | 145-146 | 25 |
| 37 | (L)-NHCH(CH(CH₃)CH₃)CO₂CH₃ | 580.63 | 191-192 | 45 |
| 38 | (D)-NHCH(CH(CH₃)CH₃)CO₂CH₃ | 580.63 | 192-193 | 40 |
| 39 | (L)-NHCH(C₆H₅)CO₂CH₃ | 648.66 | 160-161 | 32 |
| 40 | (D)-NHCH(C₆H₅)CO₂CH₃ | 648.66 | 165-166 | 35 |
| 41 | (L)-NHCH(CH₂C₆H₅)CO₂CH₃ | 676.25 | 191-192 | 42 |

### Example 45

### Pharmacological Experiment

Telomeric repeat amplification protocol (TRAP) is used in this example.

TRAP Introduction:

This protocol describes a semi-quantitative in vitro assay to detect telomerase activity. The method can be divide into two parts: (1) Telomerase elongated the sequence of telomere (TSG4 primer: 5′ GGG ATT GGG ATT GGG ATT GGG TT 3′); (2). Telomerase elongated product (CX primer: 5′CCCTTA CCCTTA CCCTTA CCCTAA3′) was replicated by PCR. When the compound expressed telomerase activity, the PCR would fail. Internal control is introducing a 36 bases oligo-nucleotides (TSNT: 5′ AAT CCG TCG AGC AGA GTT AAA AGG CCG AGA AGC GAT 3′) into TRAP. The oligo-nucleotides could be involved in the PCR of TRAP as TS primer but the NT primer (NT primer: 5′ ATC GCT TCT CGG CCT TTT-3′) is needed.

TRAP Activity Analysis:

Loaded the 360 nM CX primer, 185 nM NT primer and 400 aM oligo-nucleotides TSNT into tube and putted the wax cube (PERKIN ELMER AmpliWax PCR Gem 50) into the tube. Changed the temperature of the tube to 90° C. for 10 min, 72° C. for 3 min, 50° C. for 1 min, 20° C. for 1 min by PCR heating machine. When the temperature cooled to 4° C., took the wax cube out.

Mixed 4 μl cell extract which contains about 0.5˜2 μl cell extract protein (equal to cell extract of 10³˜10⁴ cells) with 50 μl reacting agent which contains 50 μM dNTP, 3000 cpm above taq TS prime, 360 nM non-taq TS primer, 1 μg Taq polymerase and T-PCR buffer (10×T-PCR buffer: 200 mM Tris, 15 mM MgCl2, 680 mM KCl, 0.5% Tween 20, 10 mM EGTA, pH 8.3). The distilled water was treated by 0.1% DEPC (USB) for 24 hours to eliminate the RNase in the water to avoid the interference.

Loaded the cell extract and reacting reagent into 0.2 ml PCR tube at 30° C. for 30 min to elongate the TSG4 primer by telomerase in the cell extract. Heated the mixture to 94° C. for 3 min, 94° C. for 30 sec, 50° C. for 30 sec, 72° C. for 1 min as a cycle, repeat the cycle for 39 times. The final cycle is 94° C. for 30 sec, 50° C. for 30 sec, 72° C. for 1 min to end the reaction. Added 5 μl mg/ml RNase A into the negative control of TRAP analysis.

Mixed 50 n1 of mixture after PCR and 9 n1 gel-filling buffer (6× Gel-Loading buffer: 0.25% bromophenol blue [symbol] 0.25% xylene cyanol [symbol] 30% glycerol in H2O). Added 15 μl of the gel-filling mixture in 8% TBE gel electrophoresis (acryamide:bis-acrylamide=19:1). Performed gel electrophoresis gel-eletro by 125 volt for 2 hours. The gel was stained and visualized by UV light, then analizied the result.

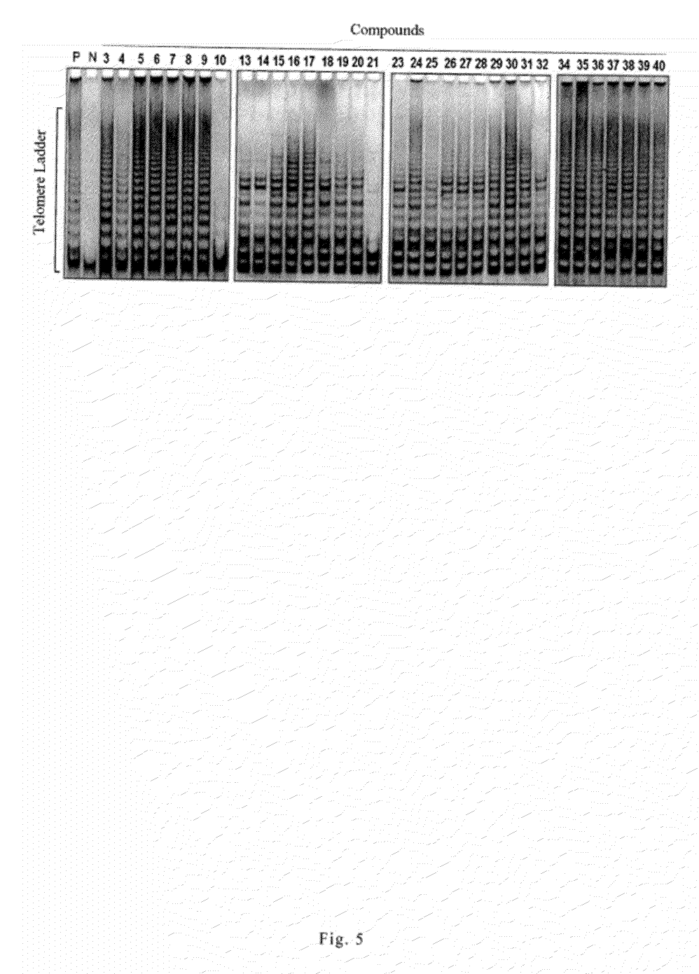

The result of compounds 3˜40 TRAP analysis was shown in FIG. 5.

The Result of TRAP Analysis:

Telomerase products were resolved by 10% polyacrylamide gel electrophoresis and visualized by staining with SYBER Green. As a source of telomerase, the total cell lysates derived from lung cancer cell line H1299 cells were used. Protein concentration of the lysates was assayed using Bio-Rad protein assay kit using BSA standards. In TRAP analysis, the TSG4 primer would form a special structure of G-quadruplex because of the sequence. The aim of the present invention is to stabilize the structure to inhibit the telomerase activity; however, the telomerase inhibiting activity has not been confirmed by TRAP analysis. Therefore, selecting the compounds which can inhibit the telomerase is the target. In FIG. 5, positive control (P) is performed by water, and negative control is performed by 5 μl 0.1 mg/ml RNase A (CLONTECH). There were many segments of telomer in positive control, but the segments were not shown in negative control. Three different concentrations of compounds were performed. The result showed 100 μM of Compound 10 and 100 μM of Compound 21 have telomerase inhibition activity.

Although the present invention has been described in terms of specific exemplary examples, it will be appreciated that the examples disclosed herein are for illustrative purposes only and various modifications and alterations might be made by those skilled in the art without departing from the spirit and scope of the invention as set forth in the following claims.

What is claimed is:

1. An anthraquinone-based compound represented by compound (I) 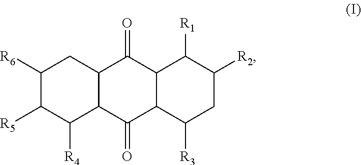
wherein the R1, R2, R3, R4, R5 or R6 is selected from the group consisting of H, amide, amino-amide, acyl, chloroacetamide, chloropropionamide, a substituted acetamide and a substituted propionamide;
wherein if the R1 and R3 are selected from the group consisting of non-substituted or substituted acetamide and non-substituted or substituted propionamido, the R2, R4, R5 and R6 are H;
wherein if the R1 and R4 are selected from the group consisting of non-substituted or substituted acetamide and non-substituted or substituted propionamide, the R2, R3, R5 and R6 are H;
wherein if the R2 and R5 are selected from the group consisting of non-substituted or substituted acetamide and non-substituted or substituted propionamide, the R1, R3, R4 and R6 are H;
wherein if the R2 and R6 are selected from the group consisting of non-substituted or substituted acetamide and non-substituted or substituted propionamide, the R1, R3, R4 and R5 are H.

2. The anthraquinone-based compound in claim 1, wherein the substituent is Cl, glycine methyl ester, alanine methyl ester, valine methyl ester, leucine methyl ester, sarcosine methyl ester, glutamic acid dimethyl ester, phenylglycin methyl ester or phenylalanine methyl ester.

3. A method of synthesizing an anthraquinone-based compound, which includes substituting R1, R2, R3, R4, R5 or R6 by non-substituted or substituted acetamide and non-substituted or substituted propionamide.

4. The anthraquinone-based compound in claim 3, wherein the substituent is Cl, glycine methyl ester, alanine methyl ester, valine methyl ester, leucine methyl ester, sarcosine methyl ester, glutamic acid dimethyl ester, phenylglycin methyl ester or phenylalanine methyl ester.

5. A composition for inhibiting the telomerase activity, which includes an effective dose of anthraquinone-based compound in claim 1 and a pharmaceutical acceptable adjuvant.


SEQUENCE LISTING (4 sequences)

SEQ ID NO: 1 - DNA, 23 bp, Artificial Sequence
  TSG4 primer
gggattgggattgggattgggtt

SEQ ID NO: 2 - DNA, 24 bp, Artificial Sequence
  CX primer
cccttacccttacccttaccctaa

SEQ ID NO: 3 - DNA, 36 bp, Artificial Sequence
  TSNT
aatccgtcgagcagagttaaaaggccgagaagcgat

SEQ ID NO: 4 - DNA, 18 bp, Artificial Sequence
  NT primer
atcgcttctcggcctttt